(19) United States
(12) Patent Application Publication
(10) Pub. No.: US 2013/0236491 A1
(43) Pub. Date: Sep. 12, 2013

(54) SPHERICAL NANO AND MICROPARTICLES DERIVED FROM PLANT VIRUSES FOR THE DISPLAY OF FOREIGN PROTEINS OR EPITOPES

(71) Applicants: Iosif Grigorievich Atabekov, Moscow (RU); Olga Viacheslavovna Karpova, Moscow (RU); Mikhail Petrovich Kirpichnikov, Moscow (RU); Nikolay Alexandrovich Nikitin, Moscow (RU); Sergey Nikolaevich Chirkov, Moscow (RU); Ekaterina Alexeevna Trifonova, Moscow (RU); Anna Alexandrovna Sheveleva, Moscow (RU); Marina Vladimirovna Archipenko, Moscow (RU)

(72) Inventors: Iosif Grigorievich Atabekov, Moscow (RU); Olga Viacheslavovna Karpova, Moscow (RU); Mikhail Petrovich Kirpichnikov, Moscow (RU); Nikolay Alexandrovich Nikitin, Moscow (RU); Sergey Nikolaevich Chirkov, Moscow (RU); Ekaterina Alexeevna Trifonova, Moscow (RU); Anna Alexandrovna Sheveleva, Moscow (RU); Marina Vladimirovna Archipenko, Moscow (RU)

(21) Appl. No.: 13/877,306

(22) PCT Filed: Feb. 15, 2011
(86) PCT No.: PCT/RU11/00081

§ 371 Date: May 14, 2013

(30) Foreign Application Priority Data
Dec. 9, 2010 (RU) 2010150332
Dec. 9, 2010 (RU) 2010150333
Dec. 9, 2010 (RU) 2010150334

(51) Int. Cl. C07K 14/08; A61K 39/39

(52) U.S. Cl. CPC C07K 14/08; A61K 39/39
USPC 424/196.11; 530/402; 530/409; 435/188; 530/405

(57) ABSTRACT

A novel type of particle platform for application in biotechnology is provided by the present invention. Said platforms comprise RNA-free particles generated by thermal denaturation and structural remodeling of helical plant viruses. Tobamovir-uses and, in particular, tobacco mosaic virus (TMV) coat protein (CP) subunits, denatured, at high temperatures are specifically self-assembled by two-stage assembly into the spherical particles (SPs) of similar shape and varying size including nanoparticles (SNPs) with a diameter up to 100-150 nm and spherical microparticles (SMPs) with a diameter up to 800 nm and more. The size of said SPs depends on the virus concentration used and, therefore can be controlled. Said SPs are biologically safe, highly stable and highly immunogenic. Said SNPs and SMPs are structurally distinct from viruses presently known. They are unique, having no protein nano-particle analogs in the nature. Said particles can be produced by the native virus and also by different forms of viral CP lacking RNA. The SPs can be generated by thermal denaturation and structural remodeling of different helical plant viruses belonging to genera Tobamovirus, Hordeivirus and family Flexiviridae. The invention relates to the creation of functionally active compostions on the base of said SP-platforms for use in medicine, veterinary, virology, immunology and diagnostics.

In accordance with this aspect of the present invention, several immunogenic compositions were obtained comprising SP platform and linked to the SP surface foreign full-size proteins, including green fluorescent protein, coat protein of PVX or epitopes of several different viruses (e.g. human influenza A virus and rubella virus epitopes). The procedure of in vitro assembly of said type of composition took about one hour. Apparently, the nanocomplexes of SNP/SMP with foreign antigens/epitopes could be regarded as candidate nanovaccines. In accordance with another aspect of the present invention, we found that the TMV-generated SPs can be used as immunological booster or adjuvant stimulating the immune response of animals by parenteral immunization.

### TECHNICAL FIELD

The present invention relates to the field of nanocomplexes and microcomplexes production by introducing in biotechnology a novel type of particle platform for assembly of biologically active compositions, and using said compositions in medicine, veterinary, virology, immunology and diagnostics.

### BACKGROUND ART

Said particle platforms comprise spherical particles (SPs) of different size including nanoparticles (SNPs) or microparticles (SMPs) obtained by thermal denaturation and structural remodeling of a native helical plant virus particles or RNA-free coat protein (CP) isolated from the native virus. More precisely, said SPs comprise thermally denatured and specifically assembled into spherical particles viral coat protein subunits. SPs are completely biologically safe, highly immunogenic, stable, and inexpensive. The size of SPs depends on virus concentration and, therefore can be controlled.

SNP/SMP are unique and have no structural analogs among viruses and particle platforms. Therefore, SNP/SMP particle platforms are radically new.

In accordance with another aspect, the present invention directly relates to the field of in vitro assembly of biologically active SP-based compositions, in particular, formation of immunogenic complexes comprising SP particle platforms with linked to their surface foreign antigen/epitopes. Said SP platforms were used in the present invention for production of nano- and microcompositions with foreign proteins and viral epitopes.

The method of compositions assembly in vitro involves binding of nano-targets of interest to the SPs surface. The primary targeting of a substance of interest to SP surface is based on non-covalent (such as, electrostatic, hydrophobic) bonding. Subsequent fixation and stabilization of said compositions is attained by formaldehyde treatment.

In accordance with this aspect of the invention, we describe here the method for fast production of immunogenic compositions by in vitro assembly of foreign antigenes/epitopes on the surface of SP-platform. In all types of immunogenic compositions provided by the present invention, the antigen/epitope molecules linked to SPs surface reacted efficiently with homologous antibodies.

Demonstrating the induction of immune responses is a key step in developing new vaccines. In accordance with this aspect of the present invention, the compositions were made comprising highly immunogenic complexes of particle SP-platform covered with foreign antigens/epitopes of viral origin. Said antigens/epitopes were packed on the surface of all SPs used in experiment. Said complexes can be considered as candidate vaccines.

Apparently, said SPs-based compositions can be used in the field of vaccines production. In addition, said complexes can be used for production of antibodies to viruses or other pathogens in cases when the preparative isolation of said antigens is complicated or the virus yield is low for production of antibodies in amounts required. In particular, SPs carrying the foreign antigens/epitopes on their surface could be used as inducers of polyclonal or monoclonal antibodies production for purposes of diagnostics.

In accordance with another aspect, the present invention directly relates to the field of stimulation of the immune response by vaccine/adjuvants. We found that TMV-generated SPs could be used as immunological booster or adjuvant stimulating the immune response by parenteral immunization of animals. In particular, the immune response (IR) to compositions “SP-foreign antigen” or to the mixtures “SP+foreign antigen” was stimulated markedly more than in control (IR to foreign antigen alone).

Considering that in vitro assembled SNP/SMP are unique and there are no structural analogs of SPs among viruses and biogenic particle nanoplatforms, it must be taken as proved that any type of composition based on SP platform (e.g. SP complex with foreign antigenes or epitopes) should be considered as radically new.

The technical and scientific terms used herein have the same meaning as commonly used in appropriate ordinary skill of the art.

As used herein, the terms “particle platform” or “nanoplatform”, as well as “carrier” refer primarily to viruses and, most particularly, to described here spherical nanoparticles (SNPs) with the size up to 150 nm.

The term “SNP-monomer” as used herein, refers to SNP with diameter close to that generated by individual TMV particle (53 nm).

As used herein, the term “spherical microparticles” (SMPs) refer to spherical particles of larger size (200-800 nm and more).

As used herein, the term (SPs) refers to both types of spherical particles (SNPs and SMPs).

As used herein, the term “mini SNP” refers to a SNP smaller than 35-40 nm generated by RNA-free TMV proteins.

As used herein, the term “irregular particles of varying size and shape” (IPs) refers to a separate subclass of platform-particles that comprise the particles of irregular shape and varying size obtained by heating the virus at about 90° C. Therein, IPs can be converted into mature SPs by heating at 98° C.

The terms “unrelated”, “foreign” (e.g. “foreign epitopes”, “epitopes of a pathogen” or “foreign protein” antigenically unrelated to that of SNP/SMP), as used herein, refer to the proteins/epitopes of interest, encoded usually (but not necessarily) not by the virus-donor of SPs, but by another virus or by alternative pathogen, different from viruses (e.g. bacteria, fungi e.t.c.).

As used herein, the term “antigen” refer to the foreign protein (immunogen) used for the SP-protein composition assembly.

As used herein, the terms “epitope” and “antigenic determinant” refer to short domains (in the present invention comprising 12-32 amino acids) of a foreign protein used for the SP-epitope complex assembly. In principal, the size of the epitope used could vary considerably.

The term “immunogenic”, as used herein, refers to the ability of SPs or “SP-antigen/epitopes” complexes to induce antibodies production in animals.

The terms “booster” and “adjuvant”, as used herein, refer to the substances having immunopotentiating or adjuvant activity. In this invention, we showed that SPs generated by TMV heating and denaturation enhanced a humoral immune response in animals.

As used herein, the terms “rod-shaped” and “filamentous” refer to helical viruses possessing a translational-rotational symmetry. The outer shell (capsid) of helical viruses consists of CP subunits assembled in a helical nanoparticle.

Several helical plant viruses were used in the present invention. In particular, the best studied common strain of tobacco mosaic virus (TMV) and several other members of the genus Tobamovirus were used.

TMV.

The TMV particles are rod-like of 18 nm diameter and 300 nm modal length. They consist of 2130 identical 17.5 kDa protein subunits closely packed by hydrophobic bonds into a rigid tube. The subunits are helically arranged with a pitch of 23 Å around a cylindrical canal of 20 Å radius. The RNA about 6,400 nucleotides long is intercalated between the protein turns at a radius of 40 Å and follows the helix of protein subunits (for reviews, see Klug A. (1999) The tobacco mosaic virus particle: structure and assembly. Philos. Trans. R. Soc. 354, 531-536; Butler P. J. G. (1999) Self-assembly of tobacco mosaic virus: the role of intermediate aggregate in generating both specificity and speed. Philos. Trans. R. Soc: 354, 537-550; Zaitlin M. and Israel H. W. (1975) Tobacco mosaic virus (type strain). C.M.I./A.A.B. Descriptions of Plant Viruses No 151).

An important feature of viruses is the possibility of the reversible dissociation of virions into CP and nucleic acid, with subsequent self-assembly of viral nanostructures. Thus, TMV can be disassembled into protein subunits with subsequent reassembly (reconstitution) of viral particles in vitro from nucleic acid and coat protein. As a result, the structure of viruses can be reassembled and their biological activity can be restored (for reviews, see Fraenkel-Conrat H. and Singer B. (1999) Virus reconstitution and the proof of the existence of the genomic RNA. Philos. Trans. R. Soc. 354, 583-586; Butler P. J. J. and Klug A. (1978) The assembly of a virus. Sci. Am. 239, 62-69; Klug A. (1999) The tobacco mosaic virus particle: structure and assembly. Philos. Trans. R. Soc. 354, 531-536). Atabekov J. G., Novikov V. K., Vishnichenko V. K., Kaftanova A. S. (1970). Some properties of hybrid viruses reassembled in vitro. Virology 41, 519-542).

The self-assembly (repolymerization) of a low-molecular-weight CP can also take place in the absence of RNA. The repolymerization of CPs proceeds in a stepwise manner with the formation of a series of intermediate protein aggregates of increasing size and leads eventually to the assembly of virus like particles (VLPs) whose length is unrestricted because of the absence of RNA.

In the absence of nucleic acid the viral coat protein may be assembled into several types of aggregate. At pH near 8.0 and low ionic strength, mixture of 4S aggregates (monomers and two-layer trimers), called A-protein, is formed (Schramm G. and Zillig W. (1955) Über die Struktur des Tabakmosaikvirus. IV. Mitt.: Die Reaggregation des nucleinsäure-freinen Proteins. Z. Naturforsch. 10b, 493-499; Lauffer M. A. and Stevens C. L. (1968) Structure of the tobacco mosaic virus particle; polymerization of tobacco mosaic virus protein. Advan. Virus Res. 13, 1-63; Butler, P. J. J., and Klug, A. (1978) The assembly of a virus. Sci. Am. 239, 62-69; Butler P. J. G. (1999) Self-assembly of tobacco mosaic virus: the role of intermediate aggregate in generating both specificity and speed. Philos. Trans. R. Soc. 354, 537-550).

The predominant aggregate at neutral pH and low ionic strength is a 20S two-layer disk-like structures. In addition to the two-layer 20S disks the stacked disk aggregates could be produced (Diaz-Avalos R. and Caspar D. L. D. (1998) Structure of the stacked-disk aggregate of tobacco mosaic virus protein. Biophys. J. 74, 595-603).

At pH values below about 6.5, TMV CP can be repolymerized into long VLPs structurally similar to native virions (Caspar, D. L. D. (1963) Assembly and stability of the tobacco mosaic virus particle. Adv. Virus Res. 18, 37-118; Anderer, F. A. (1963). Recent studies on the structure of tobacco mosaic virus. Adv. Protein Chem. 18, 1-36; Butler P. J. G. (1999) Self-assembly of tobacco mosaic virus: the role of intermediate aggregate in generating both specificity and speed. Philos. Trans. R. Soc. 354, 537-550; Namba K., Pattanayek R., and Stubbs G. (1989) Visualization of protein-nucleic acid interactions in a virus. Refined structure of intact tobacco mosaic virus at 2.9 Å resolution by X-ray fiber diffraction. J. Mol. Biol. 208, 307-325; Butler, P. J. J., and Klug, A. (1978) The assembly of a virus. Sci. Am. 239, 62-69).

The structure of TMV coat protein subunits has been studied in sufficient detail, which allows the positions of various amino acids to be localized on the capsid surface and inside the axial channel of virions (Stubbs G. (1999) Tobacco mosaic virus particle structure and the initiation of disassembly. Philos. Trans. R. Soc. London B. 354, P. 551-7).

It has been known that TMV is very heat-stable: some infectivity is retained even after 10 min exposure of infectious sap at over 90° C. Lauffer M. A., and Price W. C. (1940, Thermal denaturation of tobacco mosaic virus. J. Biol. Chem. 133, 1-15) found that heat inactivation of TMV is closely associated with CP denaturation. More than 50 years ago it has been reported (Hart R. G. 1956. Morphological changes accompanying thermal denaturation of TMV. Biochim. Biophys. Acta 20, 388-389) that heating led to swelling of TMV particles and that at 98° C. the rods were converted into “ball-like particles of about the same volume as the original rod”. Unfortunately, these studies were not developed later on.

In the present invention, we studied in more details the phenomenon of TMV CP thermal denaturation, the conditions for spherical particles (SPs) assembly and their properties

In particular, we found that the volume of the SPs (the “ball-like particles of Hart, 1956) varied over a wide range and not necessarily corresponded to the volume of original rod. Our data showed that the size of SPs generated upon TMV heating depended on virus concentration. The diameter of SPs obtained by heating TMV at concentrations of 0.1, 1.0, and 10.0 mg/ml varied in range of 50-160 nm, 100-340 nm, and 250-800 nm, respectively (Atabekov J., Nikitin N., Arkhipenko M., Chirkov S. and Karpova O./Thermal transition of native TMV and RNA-free viral proteins into spherical nanoparticles. (2011) J Gen Virol; 92: 453-456).

In accordance with another aspect of the present invention, the ability of SPs to serve as particle platform for production of spherical immunogenic compositions with foreign antigens and epitopes were demostraned.

In accordance with another aspect, the present invention, we found that the TMV-generated SPs could be used as immunological boosters or adjuvant stimulating the immune response by parenteral immunization of animals.

In accordance with another aspect, the present invention, showed that other helical plant viruses exhibited the abilities of SPs generating. The viruses belonging to family Flexiviridae, genus Potexviruses, (Potato virus X, PVX, Alternanthera mosaic virus, AltMV) and genus Hordeiviruses (Barley stripe mosaic virus, BSMV) generated SPs on heating. The members of family Flexiviridae represent flexible filamentous virions with a helical structure and a length of 515 at a diameter of 13.5 nm.

PVX.

About 1300 identical CP subunits form a polar PVX helix with a 3.6 nm pitch. The viral RNA is confined between the turns of this helix, each turn including 8-9 CP subunits. The hollow central axial channel has a diameter of 3 nm (L. Parker, A. Kendall, and G. Stubbs (2002) Surface features of potato virus X from fiber diffraction Virology 300, 291-295; P. Tollin and H. R. Wilson (1988) in The Plant Viruses, Vol. 4: The Filamentous Plant Viruses, Ed. by R. G. Milne (Plenum, New York), p. 51; J. G. Atabekov, E. N. Dobrov, O. V. Karpova, and N. P. Rodionova (2007). Potato virus X: structure, disassembly and reconstitution. Mol. Plant. Pathol. 8 (5), 667-675). The RNA of PVX consists of five genes (K. G. Skryabin, S. Yu. Morozov, A. S. Kraev, M. N. Rozanov, B. K. Chernov, L. I. Lukasheva, and J. G. Atabekov (1988). Conserved and variable elements in RNA genomes of potexviruses. FEBS Lett. 240, 33-40), PVX was the first filamentary virus reconstituted from its CP and RNA (Kaftanova A. S., Kiselev N. A., Novikov V. K., Atabekov J. G. (1975) Structure of products of protein reassembly and reconstitution of potato virus X. Virology 65, 283-287). Optical diffraction patterns confirmed the structural identity of native and repolymerized viral particles. Using atomic force microscopy (AFM) and electron microscopy data, it was recently shown that the primary products of in vitro PVX self-assembly represent VLP nanoparticles with the 5′-ends of RNA protected by CP, appearing as single-tailed particles with a free RNA tail and a helical 5′-proximal CP head (O. V. Karpova, O. V. Zayakina, M. A. Arkhipenko, E. V. Sheval, O. I. Kiselyova, V. Yu. Polyakov, I. V. Yaminskii, N. P. Rodionova and J. G. Atabekov (2006) Potato virus X RNA-mediated assembly of single-tailed ternary ‘coat protein-RNA-movement protein’ complexes. J. Gen. Virol. 87, 2731-2740).

AltMV potexvirus. (Ivanov P. A., Mukhamedzhanova A. A., Smirnov A. A., Rodionova N. P., Karpova O. V., Atabekov J. G. (2010). The complete nucleotide sequence of Alternanthera mosaic virus infecting Portulaca grandiflora represents a new strain distinct from phlox isolates. Virus Genes, (2010). DOI: 10.1007/s11262-010-0556-6). In the present invention we showed that filamentous potexviruses PVX and AltMV could be also conversed into SPs by heating.

BSMV.

The viruses belonging to the genus Hordeiviruses have nonflexible rod-shaped virions with a helical structure. Three main components have a length of about 140, 130, and 110 nm at a diameter of 18 nm. The BSMV capsid consists of identical CP subunits with a molecular weight of 23 kDa arranged in a helix with a pitch of 2.5-2.6 nm and an internal channel diameter of 3.4 nm (J. G. Atabekov and V. V Dolja (1986), in The Plant Viruses, Vol. 2: The Rod-Shaped Plant Viruses, Ed. by M. H. V. Van Regenmortel and H. Fraenkel-Conrat (Plenum, New York), p. 397). The polymerization of the BSMV coat protein in vitro proceeds in a stepwise manner with the formation of a series of aggregates of increasing size (10, 20, and 30 S) (J. Atabekov, S. Dementyeva, N. Schaskolskaya, and G. Sacharovskaya (1968) Serological study on barley mosaic virus protein polymerization. II. Comparative antigenic analysis of intact virus and some stable protein intermediates. Virology 36, 601-612; J. G. Atabekov, V. K. Novikov, N. A. Kiselev, A. S. Kaftanova and A. M. Egorov (1968) Stable intermediate aggregates formed by the polymerization of barley stripe mosaic virus protein. Virology 36, 620-638; N. A. Kiselev, D. J. DeRosier and J. G. Atabekov (1969) A double-helical structure found on the re-aggregation of the protein of barley stripe mosaic virus. J. Mol. Biol. 39, 673-674). In the present invention we showed that rodlike hordeivirus could be conversed into SPs by heating.

Nanocompositions on the Base of Chimeric Virions and VLPs.

Data available on the CP structure of plant viruses make it possible to use gene engineering methods for the directed attachment (fusing in frame) of a target epitope polypeptide to the C-, N-terminal or other amino acids localized on the surface of the given viral particle. Multisubunit nanoparticles carrying foreign epitopes on their surface may be obtained either by in vitro assembly of VLPs from chimeric subunits (“CP-foreign epitope”) either produced by genetically modified full-length virus with the coat protein gene fused to a foreign epitope (McCormick A. A. and Palmer K. E. (2008) Genetically engineered Tobacco mosaic virus as nanoparticle vaccines. Expert Rev Vaccines. 7(1), 33-41; Steinmetz N. F., Lin T., Lomonossoff G. P. and Johnson J. E. (2009) Structure-based engineering of an icosahedral virus for nanomedicine and nanotechnology. Curr. Top. Microbiol. Immunol. 327, 23-58).

In both cases a foreign epitope is covalently fused to CP subunit by genetic engineering. Numerous examples are known when a modification of viral coat protein was comprised by fusion in frame of the viral CP subunits with a foreign antigen/epitope. Recently TMV and some other viruses have been used as platforms adapted for vaccines development (Bendahmane M., Koo, M., Karrer, E., Beachy R. N. (1999) Display of epitopes on the surface of tobacco mosaic virus: impact of charge and isoelectric point of the epitope on virus-host interactions. J. Molec. Biol. 290, 9-20; Palmer K. E. et al., (2006). Protektion of rabbits against cutaneous papillomavirus infection using recombinant tobacco mosaic virus containing L2 capsid epitopes 2006, Vaccine, 24, 5517-5525; McCormick A. A., Corbo T. A, Wykoff-Clary S., Nguen L. V., Smith M. L., Palmer K. E. and Pogue G. P. (2008) TMV-peptide fusion vaccines induce cell-mediated immune responses and tumor protection in two murine models. Vaccine 24, 6414-6423).

An important direction in realization of the principles outlined above is the development of vaccines using self-replicating recombinant particles carrying the antigen determinant (epitope) of a foreign pathogene or any other functional polypeptide on the surface (S. Werner, S. Marillonnet, G. House, V. Klimyuk, and Yu. Gleba (2006) Immunoabsorbent nanoparticles based on a tobamovirus displaying protein A. Proc. Natl. Acad. Sci. USA 103(47), 17678-17683; R. Usha, J. B. Rohl, V. E. Spall, M. Shanks, A. J. Maule, J. E. Johnson and G. F. Lomonossoff (1993) Expression of an animal virus antigenic site on the surface of a plant virus particle. Virology 197, 366-374; F. R. Brennan, L. B. Gilleland, J. Staczek, V. V. Bending, W. D. Hamilton, and H. E. Gilleland (1999). A chimeric plant virus vaccine protects mice against a bacterial infection. Microbiology (Reading, UK) 145, 2061-2167; F. R. Brennan, T. D. Jones, and W. D. Hamilton (2001) Cowpea mosaic virus as a vaccine carrier of heterologous antigens. Mol. Biotechnol. 17, 15-26; Jiang 1 et al (2006) A modified TMV-based vector facilitates the expression of longer epitopes in tobacco, Vaccine, 24, 109-115; Yu. Gleba, V. Klimuk, and S. Marillonet (2007) Viral vectors for the expression of proteins in plants Curr. Opin. Biotechnol. 18, 134-141.

Frequently, the workers use as nanoparticle platforms the VLPs assembled from chimeric viral CP molecules fused to the foreign antigen/epitope (J. Denis, N. Majeau, E. Acosta-Ramirez, C. Savard, M.-C. Bedard, S. Simard, K. Lecours, M. Bolduc, C. Pare, B. Willems, N. Shoukry, P. Tessier, P. Lacasse, A. Lamarre, R. Lapointe, C. L. Macias and D. Leclerc (2007). Immunogenicity of papaya mosaic virus-like particles fused to a hepatitis C virus epitope: evidence for the critical function of multimerization. Virology 363, 59-68); Turpen H. T. et al. U.S. Pat. No. 5,977,438, November 1999. Production of peptides in plants as viral coat protein fusions; Leclerc et al. U.S. Pat. No. 7,641,896 B2, Jan. 5, 2010 Adjuvant viral particle; Werner S. et al.). The aggregated chimeric recombinant core protein of hepatitis B virus was also used as nonreplicative platforms (Filette M, et al., 2005. Universal influenza A vaccine: optimization of M2-based constructs. Virology 337, 149-161).

Practical Disadvantages of Recombinant Chimeric Viruses.

1. Construction of chimeric viruses by the methods of genetic engineering is labour- and time-consuming (about 3-10 months) for modification the viral genome and creation the recombinant genome producing chimeric CP fused genetically to a foreign antigen/epitope.

2. Not infrequently chimeric viruses cannot systemically infect the plants, but are genetically unstable, i.e. they revert into the wild type or loose the foreign epitopes in the course of replication and cell-to-cell movement. It is not unusual that chimeric constructs loose the genetically fused insertions and infectivity after the first cycles of replication (Porta C., Spall V. E., Loveland J., Johnson J. T., Parker P. J., Lomonossoff G. P. 1994. Development of cowpea mosaic virus as a high yielding system for the presentation of foreign peptides. Virology, 202, 949-955; Tailor K. M., Porta C., Johnson J. T., Parker P. J., Lomonossoff G. P. 1999. Position-dependent processing of peptides presented on the surface of cowpea mosaic virus. Biol. Chem. 380, 387-392; Bong-Nam Chung, T. Canto, P. Palukaitis 2007, Stability of recombinant plant viruses containing genes of unrelated plant viruses, J. Gen. Virol. 88, 1347-1355).

Alternatively, an affinity-conjugated antigen system was provided comprising foreign antigens conjugated via affinity moieties to the native virus or to VLP assembled from the native CP (Leclerc, D. 2010, Immunogenic affinity-conjugated antigen systems based on Papaya Mosaic Virus thereof. US Patent Application Publication, 2010/0047264 A1, February 25). Linbdo J. A., Palmer K. E., Owensboro K. Y. Smith M. I. (WO 2009/0053261, 26 Feb. 2009) introduced a reactive lysine at the N-extremity of CP subunits of recombinant TMV. To visualize this biotin-modified platform, the chimeric streptavidin (SA)-GFP was made. The (SA)-GFP boung to the lysin-modified TMV allowed to obtain (SA)-GFP-decorated virus particles.

The disadvantage of affinity-conjugated antigen system is the complexity of the procedure of making two components: e.g. the biotin-decorated recombinant virus and the “SA-antigen” complex exhibiting a specific affinity to each other. The purification procedure and separation of “virus-biotin-SA-foreign protein” complex from biotin-labelled virus and SA-labelled protein is required. The limitation of affinity-conjugated antigen system is due to the fact that specific affinity exists only between two moieties: the modified platform (viral CP) and SA-antigen.

By contrast, the particle platform SPs developed in the present investigation are capable of binding various type of proteins, which can be regarded as an advantage of the virus-generated SPs as a particle platform.

It should be emphasized that in all cases where recombinant viruses have been used as platforms adapted for vaccine or other function development, the morphology and general architecture of the native virus was generally retained. In other words, the virus-like capsid served as a particle platform in these cases.

By contrast, said SNP/SMP particle platforms comprise RNA-free particles generated by thermal denaturation and structural remodeling of native viruses. Viral CP subunits denatured at high temperatures are specifically self-assembled into the spherical nanoparticles. These facts indicate that SNP/SMP provided by the present invention represent a novel type of particle platform for biotechnology.

In the present invention, the SPs assembled from thermally denatured viral CP were used for linking to their surface of foreign antigens and epitopes. SPs were shown to be advantageous for in vitro assembly of immunogenic complexes carrying on their surface one or more types of foreign epitopes. Apparently, the complexes of SPs with foreign antigens/epitopes could be regarded as candidate nanovaccines.

### DISCLOSURE OF INVENTION

The primary aim of the present invention was to provide a new particle platform for application in nanotechnology, immunology, medicine and veterinary. Said platform comprises spherical particles (SPs) of various size and similar shape obtained by short thermal denaturation and structural remodeling of helical plant viruses (most particularly, tobamoviruses) resulting in assembly of denatured CP into SPs. The SPs vary in size from SNPs (diameter 53-150 nm) to SMPs (diameter up to 800 nm and more).

In accordance with another aspect of the present invention, the method is provided for the size control of said SNPs/SMPs. We found that the size of SPs depended on virus concentration and, therefore can be controlled (FIG. 1-7) (Atabekov Joseph, Nikolai Nikitin, Marina Arkhipenko, Sergey Chirkov and Olga Karpova//Thermal transition of native TMV and RNA-free viral proteins into spherical nanoparticles. (2011) J Gen Virol; 92: 453-456),

SPs are completely dissimilar from all other types of particle nanoplatform used. The SPs are structurally unique having no analogs among viruses and other biological subjects.

The evidence is provided indicating that SP platform is highly stable: SNP/SMPs do not change their shape and size, do not fuse and do not change the state of aggregation after:

1. Repeated freezing at −20° C. and thawing;

2. Repeated heating up to 98° C. and cooling;

3. Pelleting by centrifugation at 10,000 g and resuspension;

4. Storage for at least for 6 months at 4° C.

In accordance with another aspect of the present invention, there is provided a method for immature intermediate precursors of SPs generating that comprised irregular particles of varying size and shape (IPs) (FIG. 1). The IPs can be obtained by heating TMV at 90° C. The vast majority of discrete IPs of varying size and shape were accumulated at 90° C. Most significantly, heating of these particles up to 94-98° C. resulted in transition of IPs into traditional mature spherical SNPs/SMPs. No residual IPs are revealed after TMV heating at 94-98° C., indicating that 100% of irregular particles and TMV rods are entirely converted into SPs.

In accordance with another aspect of the present invention, a comparative antigenic analyses of TMV and SP-platforms were provided. Antigenically the SP is only distantly related to TMV. The results of ELISA suggested that about 3-5% of native TMV epitopes were retained after TMV-to-SPs transition.

Conversely, the SPs assembled from A protein trimer (antigenically different from native TMV) are structurally similar and antigenically closely related to SPs generated by native virus. Apparently, a specific conformation favorable for SPs assembly (SP generating conformation) could be caused by heating of different forms of viral CP. The data presented by invention suggest that a unique SPs-generating conformation of thermally misfolded CP subunits leads to their selective assembly into SPs (J. Atabekov, N. Nikitin, M. Arkhipenko, S. Chirkov and O. Karpova//Thermal transition of native TMV and RNA-free viral proteins into spherical nanoparticles. (2011) J Gen Virol; 92: 453-456).

On the whole, (i). Said SP platforms comprise a denatured viral CP specifically self-assembled into the SNP/SMPs of varying size and the same shape upon the native virus heating, (ii). All types of SPs are water insoluble and produce either a colloidal solution or relatively stable suspension (depending on SPs size), which could be precipitated by centrifugation at 10,000×g, (iii) The sizes of SPs depend on the virus concentration used and varies from 53 nm (SNP-monomers generated by individual TMV virions) to 100-800 nm and more large SPs, (iv). The SP is biologically safe since plants and humans have no common pathogens, (v). The SPs are unusually stable, (vi). The SPs do not contain RNA, are structurally distinct from viruses presently known and have no protein nanoparticle analogs in nature, (vii). Said SPs can be produced by different helical plant viruses. More particularly, the (SPs)-generating viruses are members of Tobamovirus genus, and most particularly, the tobacco mosaic virus (TMV). In addition, the SPs can be generated by members Flexiviridae family (genus Potexvirus) and genus Hordeivirus.

Another aim of the present invention was to provide compositions comprising said SP-platforms associated with substances of interest, in particular, with foreign antigens/epitopes linked to SPs surface. In accordance with this aspect of the present invention we showed that various type of foreign proteins could be bound (absorbed) to the surface of the SPs, allowing biologically active compositions production after formaldehyde treatment.

This feature can be regarded as an advantage of the virus-generated SPs particle platform. Listed below are the antigens/epitopes which readily bound to the surface of SPs producing immunogenic compositions: green fluorescent protein (GFP), coat protein isolated from potato virus X, the N-terminal M2e epitope of transmembrane surface protein M2 of human influenza virus A, tetraepitope of influenza virus A hemagglutinine, epitope of Rubella virus glycoprotein E1. Furthermore, the “SP-antigen/epitope” complexes generated in the present invention were stabilized by 0.05% formaldehyde. Fluorescent microscopy revealed the said foreign antigen/epitopes linked to the surface of the said SP platform. The foreign antigens/epitopes linked to the surface of SP-platform reacted with antibodies to the respective antigen/epitope indicating that the specificity of these antigens/epitopes was not changed after their binding to the SP surface (FIGS. 9-13).

Advantages Provided by In Vitro Assembly of “SP-Antigen/Epitopes” Compositions.

The evidence provided by the present invention suggests that SP-platforms could be used for immunogenic complexes-nanovaccines in vitro assembly.

The compositions of SPs carrying foreign epitopes on their surface will have several advantages over the attenuated, chemically inactivated and subunit vaccines:

(i) this approach excludes the possibility of pathogenic reversions and recombination because SP-antigen compositions are assembled from genetically inert components;

(ii) vaccines obtained by this way on the basis of plant viruses as starting material will be safe for humans, since plants and animals have no common pathogens;

(iii) the procedure of SP-platforms production is based on heating of the virus at 94-98° C.; therefore, no additional sterilization of SPs is required;

(iv) the procedure of in vitro assembly of compositions on SP-platforms is simple and fast (about 1 h).

(v) various proteins, vaccinogens, epitopes can be used for assembly on the surface the SP-based compositions; assembly of compositions occurres due to unique ability of SPs to absorb various proteins of interest (PI) with subsequent washing of the complex by centrifugation and fixation of PI molecules on the SP surface by formaldehyde.

(vi) production of SP platform is inexpensive, since the virus donor of SP (in particular, TMV), accumulates in plant leaves at extremely high amounts. The yield of TMV may reach 10 g/kg of leaves and the procedure of TMV purification is very simple and inexpensive. The yields of majority of other helical plant viruses are also high enough.

(vii) ELISA tests demonstrated that the said compositions were highly immunogenic in mice.

(viii) It was shown that the TMV-generated SPs as well as SPs linked to antigen have the property of immunological booster or adjuvant, stimulating immune response by parenteral immunization of animals (FIGS. 15 and 16).

(ix) It is noteworthy that immune response induced by SPs was 20-times higher than that induced by native TMV (FIG. 14).

On the whole, fast, simple and widely available methods for (i) new stable, universal and biologically safe virus-generated SPs platform production and for (ii) in vitro assembly of SPs-based immunogenic compositions with adjuvant activity were developed.

### BRIEF DESCRIPTION OF DRAWINGS

FIG. 1 presents electron microphotographs illustrating irregular particles (IPs) of varying size and shape (an immature intermediate precursors of SPs generated by TMV at 90° C.). Heating of IPs up to 94-98° C. resulted invariably in their transition into mature SPs.

FIG. 2 illustrates mature SPs generated by native 0.1 mg/ml TMV at 94-98° C. Samples were treated with 2% uranyl acetate.

FIG. 3 illustrates mature SPs generated by native 1 mg/ml TMV at 94-98° C. Samples were treated with 2% uranyl acetate.

FIG. 4 illustrates mature SPs generated by native 10 mg/ml TMV at 94-98° C. Samples were treated with 2% uranyl acetate.

FIG. 5 illustrates mature SPs generated by native 0.1 mg/ml TMV at 94-98° C. Scanning electron microscopy.

FIG. 6 illustrates mature SPs generated by native 1 mg/ml TMV at 94-98° C. Scanning electron microscopy.

FIG. 7 illustrates mature SPs generated by native 10 mg/ml TMV at 94-98° C. Scanning electron microscopy.

FIG. 8 presents a schematic (not to scale) representation of the SPs generation by native TMV and by RNA-free forms of TMV protein. The numbers indicate concentrations of native TMV (at left) and RNA-free TMV proteins (at right) heated at 94° C. or 65° C., respectively. The size ranges of SPs (in nm) are indicated.

FIG. 9 presents a schematic representation of one (Ag1) and two (Ag1+Ag2) model antigens binding to SP-platform.

FIG. 10 illustrates binding of fluorescent GFP molecules to the surface of SPs nanoplatform. Fluorescent microscopy.

FIG. 11 presents a schematic representation of detection by fluorescent microscopy of antigen Ag1 linked to the surface of “SP-Ag1” complex. Significantly, the primary antibodies readily react with Ag1 molecules on the surface of SP platform.

FIG. 12 illustrates that whole surface of each of SPs is covered with fluorescent PVX CP molecules labelled with fluoresceineisotiocyanate (left); (FITC)-labelled PVX. (right) control:transmitted light. Bar, 3 μm. Laser Scanning Confocal microscopy.

FIG. 13 illustrates that polyepitopes of hemagglutinine (HA) of human influenza A virus are bound to the surface of SP-platforms. The surface of all SPs is covered with tetraepitopes consisting each of 4 conservative monoepitopes of HA. Detection with primary mice anti HA antibodies and secondary antimice antibodies conjugated to fluorofore. Control-transmitted light (left); fluorescent microscopy (right). Bar, 3 μm.

FIG. 14 comparison of mice antisera titers obtained after immunization with SPs and with native TMV.

FIG. 15 comparison of titers of mice antisera obtained after immunization with “SPs+PVX” CP mixture and with “SPs-PVX CP” compositions fixed with formaldehyde and carrying PVX CP on their surface.

FIG. 16 illustrates stimulation of immune response (adjuvant effect) of SPs by comparison of immune response of mice to the recombinant protein N1 immunized alone, to SPs+N1 mixture and to “SPs−N1” compositions stabilized by formaldehyde.

### MODES FOR CARRYING OUT THE INVENTION

It has been known that TMV is very heat-stable: some infectivity is retained even after 10 min exposure of crude infectious sap at over 90° C. It was found (Lauffer, M. A., and Price, W. C. 1940; Thermal denaturation of tobacco mosaic virus. J. Biol. Chem. 133, 1-15) that heat inactivation of TMV is closely associated with CP denaturation. More than 50 years ago it has been reported (Hart, R. G. 1956; Morphological changes accompanying thermal denaturation of Tobacco mosaic virus. Biochim. Biophys. Acta 20, 388-389) that after heating for 10 sec at 98° C. the rods were converted into “ball-like particles of about the same volume as the original rod”. The author did not consider the possibility of practical application of “ball-like particles”. Unfortunately, these studies were not developed later on.

In the present invention, we studied in more details the phenomenon of the TMV CP thermal denaturation, the conditions for spherical particles (SPs) of different size assembly, their properties as particle nanoplatforms for immunogenic compositions formation and as adjuvant enhancing the immune response in animals.

The objectives of the present invention were: (a) to study in more details the phenomenon of the TMV CP thermal denaturation and the products generated by thermal denaturation of different helical plant viruses; (b) to characterize the properties of these products including size, shape, heterogeneity, solubility in water, the degree of their stability, reversibility of denaturation, the presence of RNA and (c) the most important problems were to study (1) if the products of thermal denaturation can be used in biotechnology and, particularly in nanobiotechnology, as new type of particle platforms for nano- and microcompositions formation with biologically active substances, (2) if the said products can be generated by different helical plant viruses, (3) if the products of helical viruses thermal denaturation can be used for in vitro assembly of biologically active compositions (vaccines, for example) comprising said type of particle platforms allowing binding to their surface widely different substances, including foreign epitopes, immunogens, entire protein molecules and their aggregates, (4) if the said SP-platforms dispose of adjuvant capacity, i.e. are capable of enhancing the immune response of the animal to foreign antigens.

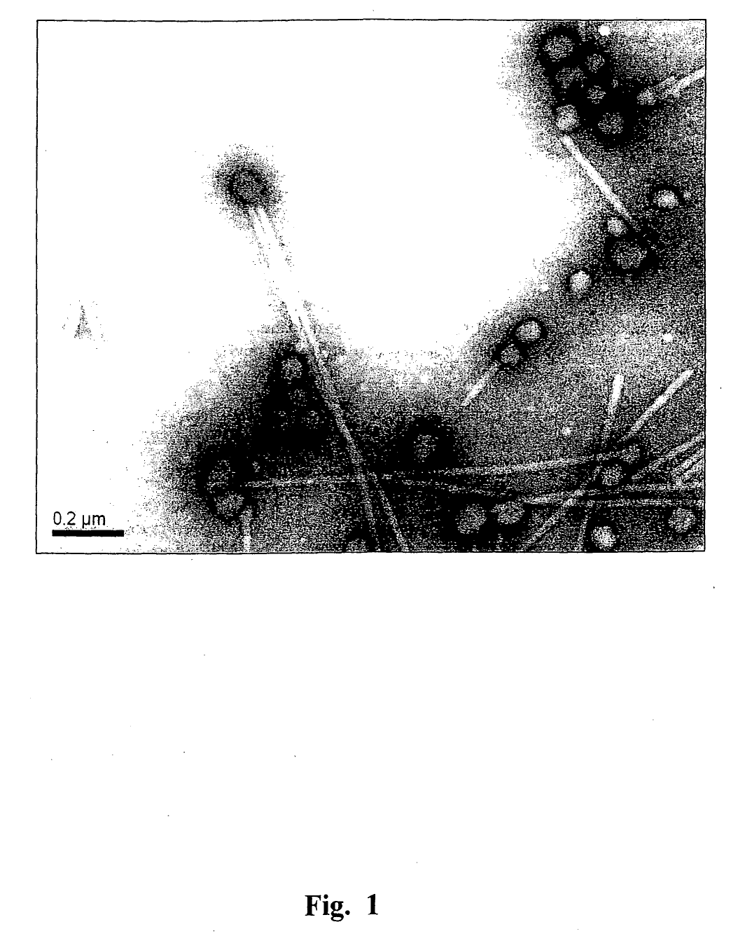

The present invention provides new information concerning the process of SPs generation. In particular, transmission electron microscopy (TEM) showed that the vast majority of particles produced at 90-92° C. (in distilled water or in 10 mM Tris-HCl buffer, pH 7.8) were not spherical, but represented irregular shape particles (IPs) of varying size and shape. Numerous discrete, separate particles of irregular shape were accumulated (FIG. 1). Most significantly, subsequent heating of IPs at 98° C. resulted in their conversion into mature spherical SPs. No residual IPs were revealed after TMV heating at 98° C. for 10 sec, indicating that 100% of IPs and TMV rods were entirely converted into SPs. Apparently, the IPs represent immature intermediate precursors of SPs produced at the first step of mature SP formation.

Our TEM studies confirmed that after heating for 10 sec at 98° C. the rods were converted into “ball-like particles” referred in the present invention as SPs. However, the SPs generated at 94-98° C. were not “about the same volume as the original rod”. By contrast, they were heterogeneous in size and, therefore the volume of SPs, varied over a wide range.

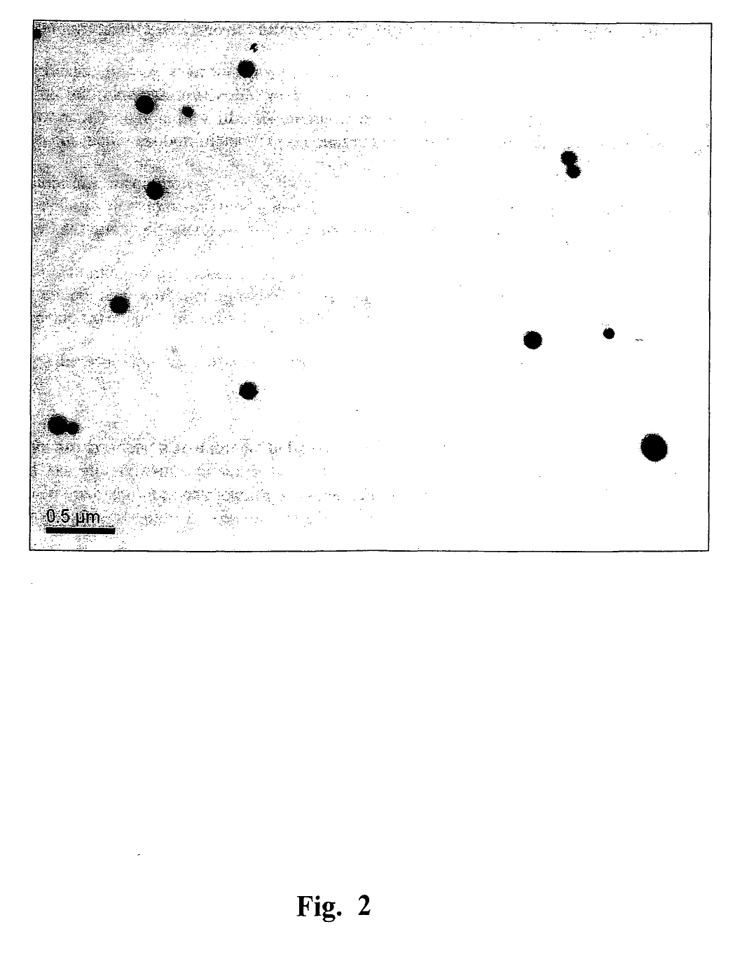

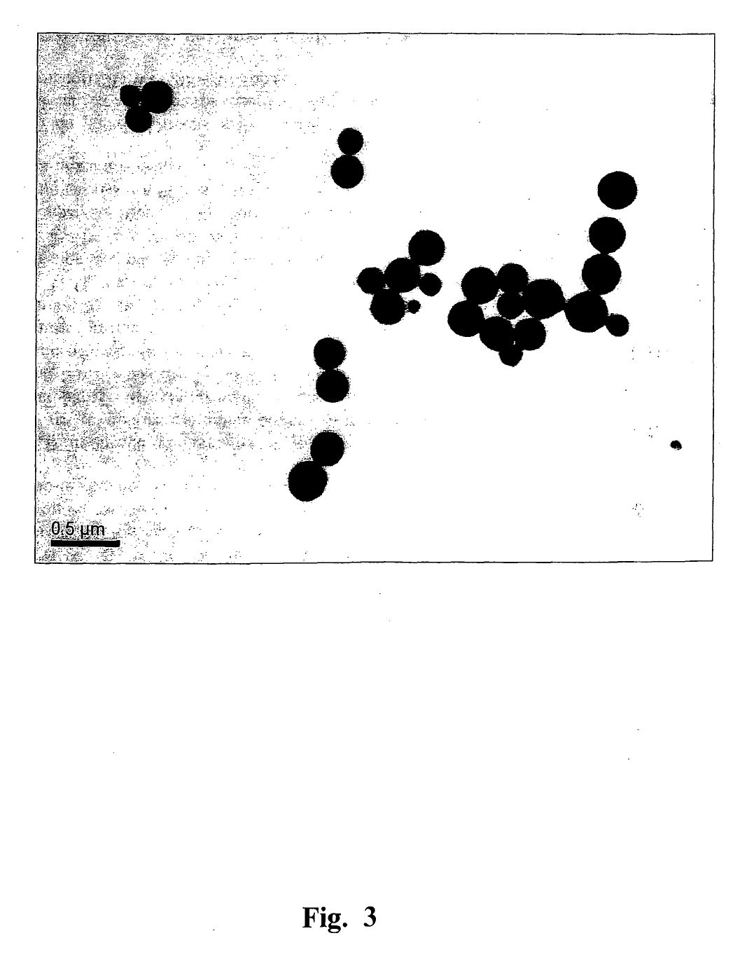

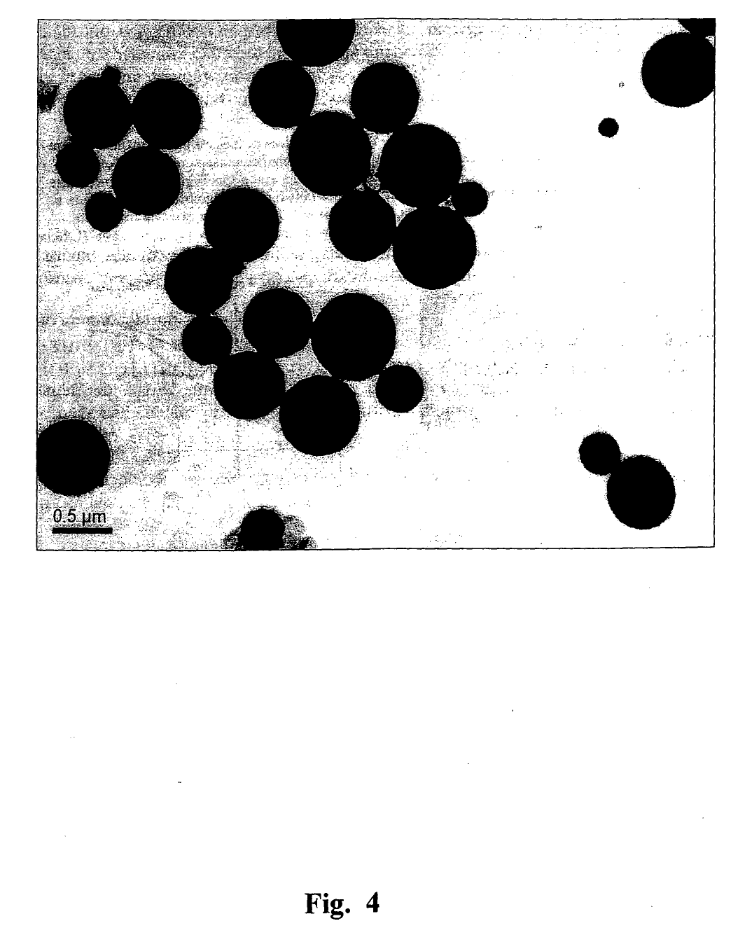

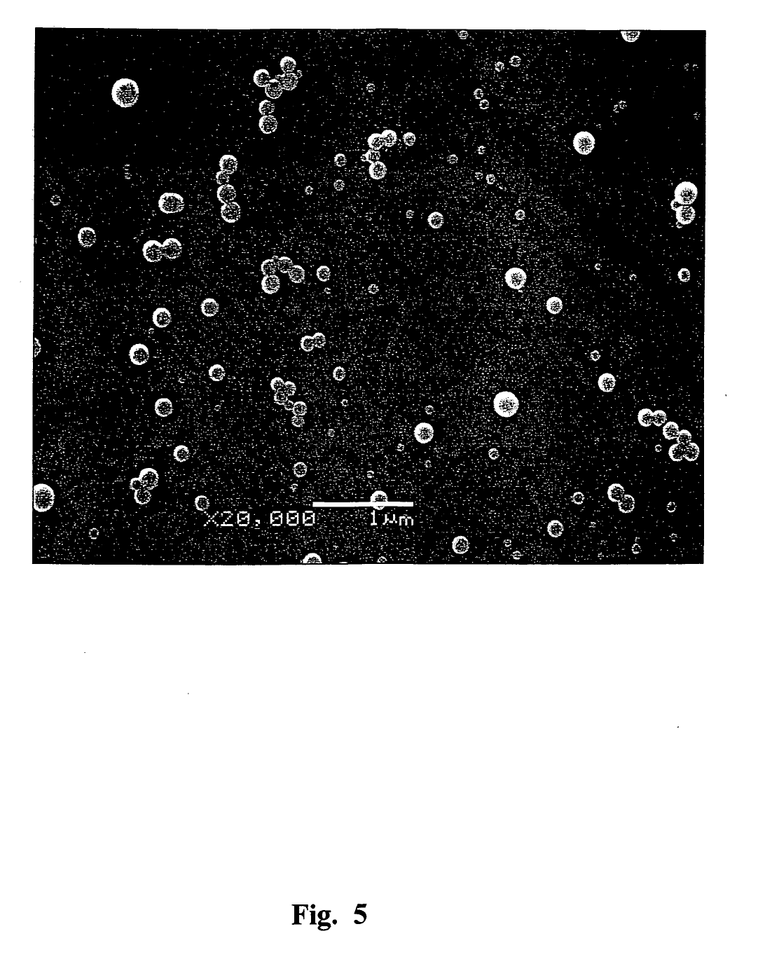

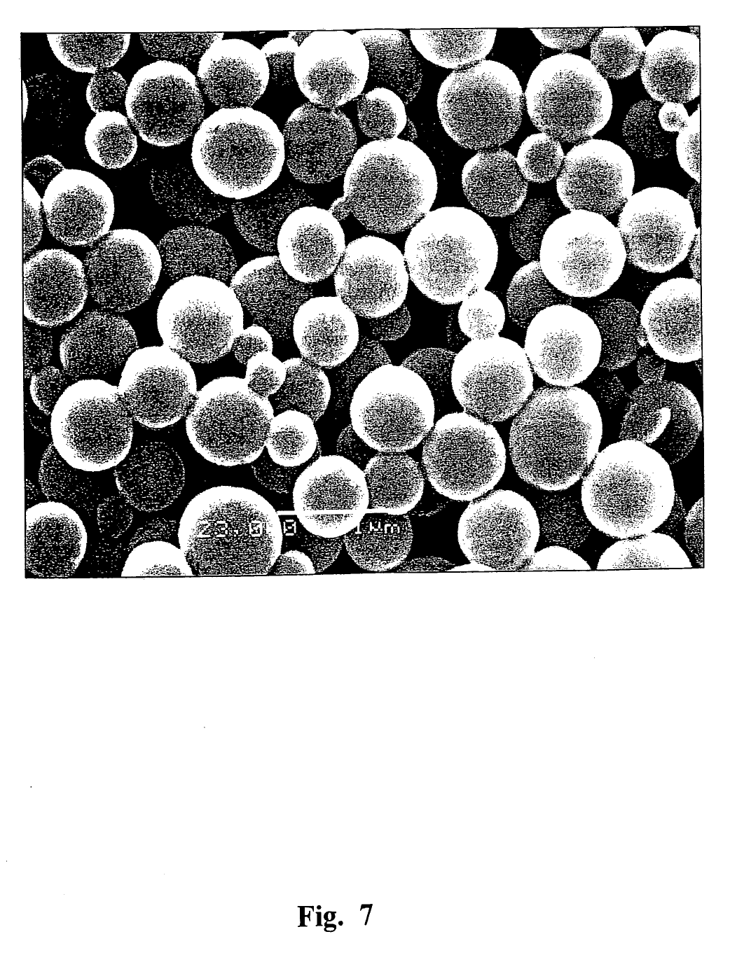

It was shown in the present invention that the size of SPs generated upon TMV heating depended heavily on virus concentration. Thus, the size of SPs generated by native TMV at concentrations of 0.1, 1.0, and 10.0 mg/ml were in range of 50-160 nm, 100-340 nm, and 250-800 nm, respectively (FIG. 2-4). FIGS. 5-7 shows that this effect was readily illustrated, by scanning electron microscopy. It could be calculated that diameter of SPs corresponding to the volume of individual TMV particle is 52.6 nm. Therefore, the SPs with diameter close to this size were referred to as TMV-generated “monomers”. Apparently, several SNP-monomers fused into the large “poly-SNPs”.

Spectrophotometry of supernatant and SPs pellet obtained after centrifugation showed that viral RNA was totally released upon SPs generation. Agarose (1%) gel electrophoresis indicated that released RNA was drastically degraded. Consequently, SPs consisted of thermally denatured CP and did not contain RNA

The mobility in SDS PAGE of TMV CP isolated from native virus and from SNPs was similar. However, the protein isolated from SNP by acetic acid dissociation could not be reassembled into any regular structure indicating that thermal denaturation was irreversible (Atabekov Joseph, Nikolai Nikitin, Marina Arkhipenko, Sergey Chirkov and Olga Karpova//Thermal transition of native TMV and RNA-free viral proteins into spherical nanoparticles. (2011) J Gen Virol; 92: 453-456),

In accordance with another aspect of the present invention, the evidence is provided indicating that SP platform is highly stable: SNP/SMPs do not change their shape and size, do not fuse and do not change the state of aggregation after:

1. Repeated freezing at −20° C. and thawing; 2. Repeated heating up to 98° C. and cooling; 3. Pelleting by centrifugation at 10,000 g and resuspension; 4. Storage for at least for 6, months at 4° C.

It worth mentioning that TMV is readily available, cheap and amenable subject for various types of study: 1) it is very stable and highly immunogenic; the purified TMV preparations retain infectivity for decades, 2) TMV can be purified by many simple procedures such as differential centrifugation, salt, isoelectric point precipitation or polyethylene glycol procedure, 3) the yields of TMV can reach the levels as high as 10 g/kg fresh tobacco leaves (Zaitlin, M., and Israel, H. W. (1975) Tobacco mosaic virus (type strain). C.M.I./A.A.B. Descriptions of Plant Viruses No 151).

It was shown in the present invention that spherical nanoparticles (SPs) could, be generated not only by native TMV rods. The said SPs of various size were readily produced by heating of different types of RNA-free TMV protein, including RNA-free helical VLPs, disk-like aggregates, A protein (monomer-trimer), and even by individual elemental TMV protein subunits. All types of SPs are water insoluble and produce either a colloidal solution or relatively stable suspension (depending on SPs size), Our results provide evidence that, upon thermal denaturation, the CP subunits acquire a specific conformation favorable for assembly into SPs (Atabekov Joseph, Nikolai Nikitin, Marina Arkhipenko, Sergey Chirkov and Olga Karpova//Thermal transition of native TMV and RNA-free viral proteins into spherical nanoparticles. (2011) J Gen Virol; 92: 453-456).

SPs Generation by RNA Free VLPs.

It has been established that polymerization of TMV A protein is endothermical, concentration-dependent, and reversible. TMV protein polymerizes when concentration and/or temperature is increased and depolymerizes when they are decreased (reviewed by Lauffer, M. A., and Stevens, C. L. (1968) Structure of the tobacco mosaic virus particle; polymerization of tobacco mosaic virus protein. Advan. Virus Res. 13, 1-63). Apparently, the RNA-free high-molecular-weight helical VLPs are less stable than native TMV and, therefore VLP preparations contain some amount of low-molecular-weight A protein, presumably due to “VLP-A protein” equilibrium.

Three concentrations (0.1, 1.0, and 10.0 mg/ml) of TMV A protein in 100 mM NaCl phosphate buffer, pH 5.8-6.0 were used here for VLPs assembly and subsequent VLP-SNP transition by heating. Generation of helical VLPs and their conversion into SNPs was detected by TEM.

The differential scanning calorimetry (DSC) showed that thermal stability of helical TMV VLPs was lower than that of native TMV (Mutombo, K, Michels B., Ott, H., Cerf, R., and Witz, J. (1992) Scanning calorimetric studies of the stability of tobacco mosaic virus and aggregates of its coat protein. Eur. Biophys. J. 21, 77-83; Orlov V. N., Kust S. V., Kalmykov V. P., Dobrov E. N., and Drachev V. A. (1998) A comparative differential scanning calorimetric study of tobacco mosaic virus and its coat protein is mutant. FEBS Lett. 433, 307-311). Therefore, we checked the possibility of VLP to SP transition at the temperature considerably lower than 94-98° C. (the temperature required for native TMV to SP transition). It is noteworthy that no VLP-SP transition was revealed at 50° C. In line with our expectations, the VLP-SP transformation readily occurred at 65° C. and higher (in the range from 65° C. to 98° C.). No residual helical VLPs or disks were revealed after heating up to 65° C.

Most significantly, two types of SPs were generated upon VLPs heating, namely, the large particles heterogeneous in size similar to those generated by native TMV and many heterogeneous SPs considerably smaller than SNP-monomer generated by TMV. By convention the SNPs smaller than 35-40 nm generated by RNA-free TMV proteins were referred to as “mini SNPs”. These observations provided the first evidence that SNPs can be generated not only by native TMV virions, but also by RNA-free CP aggregates.

SPs Generation by Disk-Like Aggregates.

Assembly of disk-like aggregates from A protein was performed in 50 mM NaCl phosphate buffer, pH 7.0 and was controlled by TEM. Three concentrations of A protein (0.1, 1.0 and 10.0 mg/ml) were used to examine the disk-to-SP transition. No SP were revealed after heating disk-like aggregates up to 50° C., whereas, the mixture of large and mini SNPs was generated at all three protein concentrations upon heating up to 65° C. or up to 98° C. (J. Atabekov, N. Nikitin, M. Arkhipenko, S. Chirkov and O. Karpova//Thermal transition of native TMV and RNA-free viral proteins into spherical nanoparticles. (2011) J Gen Virol; 92: 453-456).

SPs Generation by A Protein.

It has been shown that the equilibrium between TMV monomer (17.5 kDa subunit) and cyclical trimer (A protein) was shifted to dissociation of 4S A protein aggregate into 2.0 S monomers by lowering the concentration to 1 mg/ml, increasing the pH, decreasing the ionic strength and temperature. By this means the 4S A protein (in 10 mM KCl at pH 7.2 and 6° C.) dissociated into 1.9 S subunits when the concentration was reduced to 0.1 mg/ml (Lauffer, M. A., and Stevens, C. L. (1968) Structure of the tobacco mosaic virus particle; polymerization of tobacco mosaic virus protein. Advan. Virus Res. 13, 1-63). Therefore, we examined the possibility of SPs generation by heating A protein preparations up to 50, 65, 80, 94 and 98° C. at conditions favorable for dissociation: low ionic strength (10 mM Tris-HCl), high pH level (pH 7.8) and various concentrations 0.1, 1.0 and 10.0 mg/ml. Firstly, it was found that the low-molecular-weight A protein was capable of generating a mixture of SPs at 65° C. This was generally in line with results of differential scanning calorimetric studies of Orlov et al. (Orlov V. N., Kust S. V., Kalmykov V. P. Dobrov, E. N. and Drachev, V. A. (1998) A comparative differential scanning calorimetric study of tobacco mosaic virus and its coat protein is mutant. FEBS Lett. 433, 307-311) who did not look for SPs, but showed that melting of A protein occurs at about 40° C. Secondly, the SPs generated by A protein heating at 65° C. and 98° C. were significantly more heterogeneous than those produced by rod-like particles of native TMV indicating that a considerable amount of mini SNPs together with large-size SNPs were produced. No SPs or disks were revealed by TEM after A protein heating up to 50° C., Thirdly, no A protein to disks assembly occurred at the first stages of heating, prior to A protein denaturation. These results provided evidence that trimer of subunits or/and chemical monomers of TMV CP could be involved in SPs generation. Taken together, our data indicated that the specific helical arrangement of CP subunits was not essential for TMV CP-SP transition.

SPs Generation by Elemental CP Subunits.

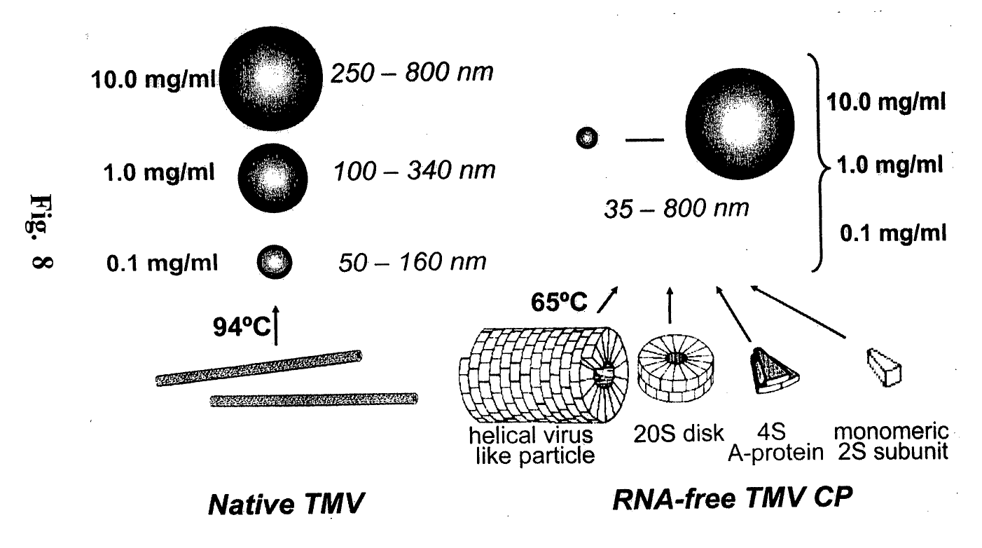

It has long been known that the ultimate elemental CP subunits (about 2.0 S) were obtained when A protein was taken at pH 13 (Anderer, F. A. (1959) Das Molekulargemicht der Peptide inheit im Protein des Tabakmosaikvirus. Z. Naturforsch. 14, 24-28; Wittmann, H, G. (1959) Darstellung and physikochemiche Peptidketten des Tabakmosaikvirus. Experientia 15, 174-175). In order to study the possibility of monomeric CP subunits transition into SPs the preparation of A protein (0.1 mg/ml) in 10 mM Tris, pH 14 was heated up to 94° C. It was found that, similarly to three types of RNA-free protein aggregates mentioned above, the TMV protein dissociated into minimal (individual) subunits was converted by heating into a mixture of mini and larger SPs. The results presented above are schematically illustrated by FIG. 8.

SPs can be Generated by Different Helical Plant Viruses.

Plant viruses of the genus Tobamovirus are similar in their virion morphology, and genome organization. In addition to common TMV four other tobamoviruses were used in experiments on SPs production, including Cucumber green mottle mosaic virus (CGMMV), Crucifer infecting TMV (crTMV or TVCV—Turnip vein-clearing virus), Tomato mosaik virus (ToMV) and Sunn-hemp mosaic virus (SHMV) (or Dolichos Enation Mosaic Virus).

Two Potexviruses (Potato virus X, PVX, and Alternanthera mosaic virus, AltMV) are also helical viruses, but unlike TMV, their particles are not rigid, but flexible, filamentous and somewhat longer than those of TMV (for review, see Kendall A., McDonald M., Bian W., Bowles T., Baumgarten S. C., Shi J., Stewart P. L., Bullitt E., Gore D., Irving T. C., Havens W. M., Ghabrial S. A., Wall J. S., Stubbs G. (2008) Structure of flexible filamentous plant viruses. J Virol. 82(19), 9546-9554).

The data provided in the present invention show that, in addition to tobamoviruses, other helical plant viruses can be structurally remodeled and converted into SPs. In particular, the SPs can be generated by thermal denaturation of other helical viruses, including the rodlike BSMV hordeivirus, and flexible PVX and AltMV potexviruses.

Antigenic Specificity of SPs

The comparative analyses showed that native TMV and SP were antigenically only distantly related. The results of ELISA suggested that about 3-5% of native TMV epitopes were retained after TMV-to-SPs transition.

Finally, antigenic specificity of SPs generated by different forms of TMV CP was examined. Namely, we compared the SPs generated by native TMV and those produced by A protein antigenically different from native TMV. It was shown that SPs generated by these two distinct forms of TMV CP were antigenically closely related. Apparently, a specific conformation favorable for SPs assembly (SP generating conformation) could be caused by heating of different forms of viral CP. (Atabekov Joseph, Nikolai Nikitin, Marina Arkhipenko, Sergey Chirkov and Olga Karpova//Thermal transition of native TMV and RNA-free viral proteins into spherical nanoparticles. (2011) J Gen Virol; 92: 453-456)

In Vitro Assembly of Immunogenic Compositions Comprising SP Platform and Foreign Proteins or Epitopes Linked to the SP Surface.

The SP nanoplatform has no analogues in nature, is stable and can be used in biotechnology for various nanocomposites formation. In particular, it was shown in the present invention that SP can serve as a universal protein platform for immunogenic compositions assembly.

A kinetic unit in viral solutions is a quite compact, closely packed nanoparticle containing a certain amount of bound water. It was demonstrated that the interaction of viral particles with water has a purely surface character (Caspar, 1963). It is probable that the thermal denaturation and misfolding of protein subunits makes the surface of SPs more hydrophobic and capable of absorbing foreign proteins.

The present invention indicates that, contrary to native TMV, the surface of SPs has high absorption capacity in respect to various proteins. This feature of SPs was illustrated by experiment where TMV and SPs were incubated with FITC-labelled CP of PVX (CP^{FITC}). The samples contained 50 μg of SP or TMV and 20 μg of fluorescent CP^{FITC}. After 15 min incubation at room temperature the samples were centrifuged at respective speeds to separate the particles from unbound CP^{FITC}. The pellets were resuspended and analysed at 495 nm (wave length of maximum absorption for FITC). It was found that more than 50% of FITC-labelled PVX CP was absorbed by SPs. By contrast, no CP^{FITC} was bound to TMV.

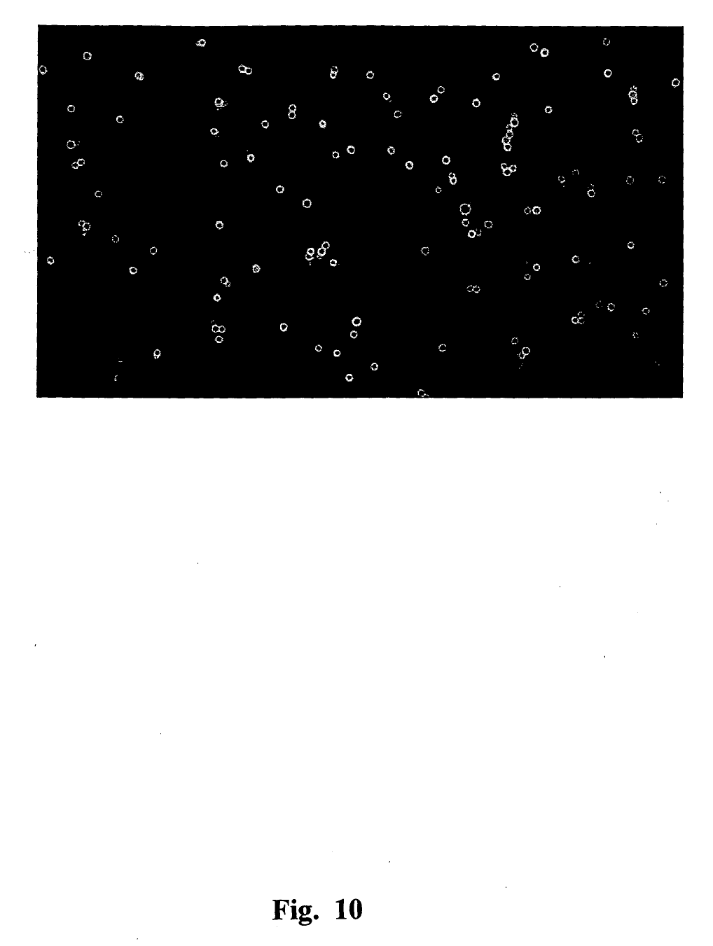

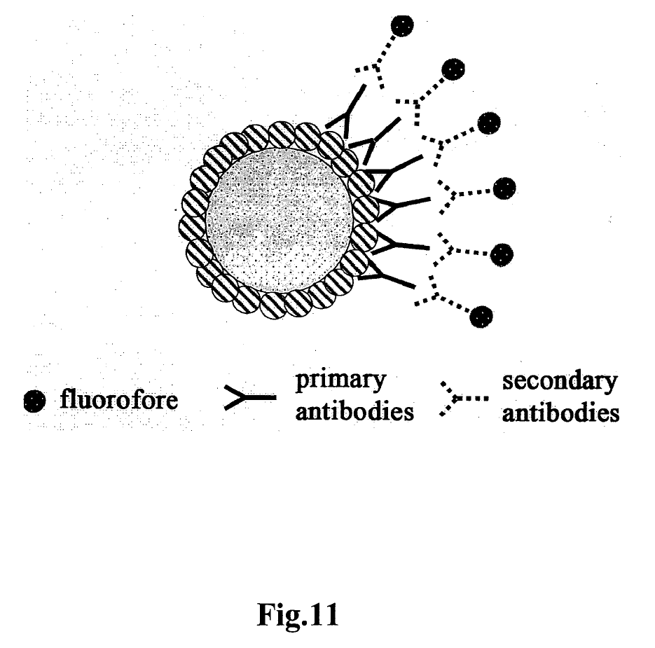

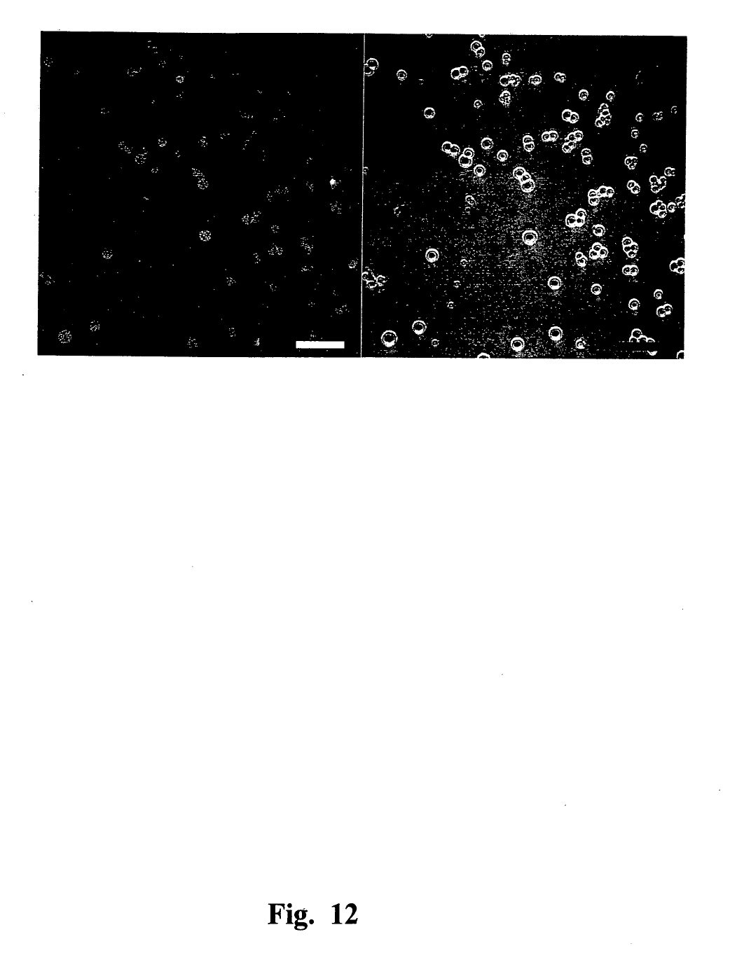

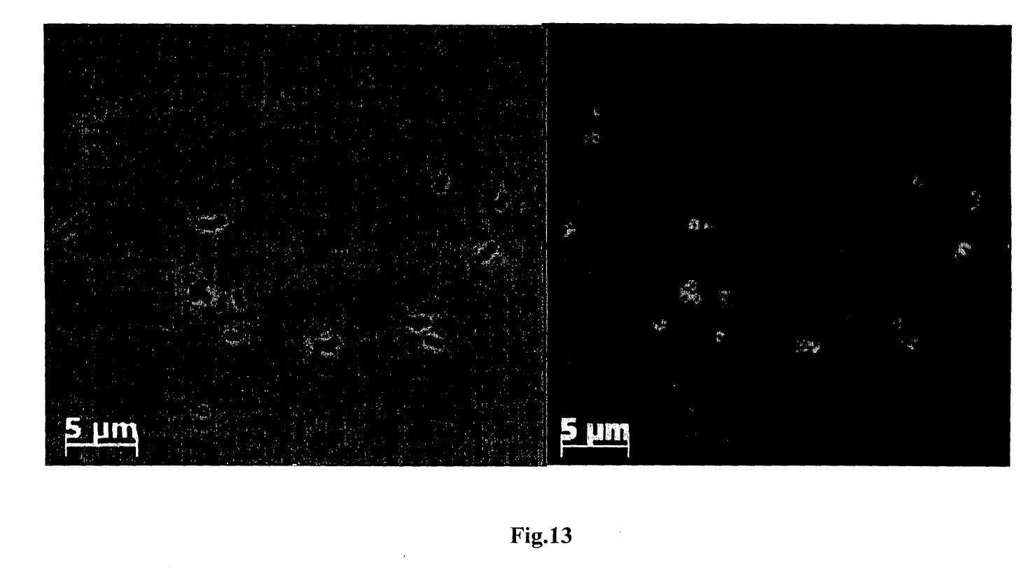

In the present invention, the SPs were used for binding to their surface of several bacterially expressed recombinant (or natural) antigens and epitopes listed below: (i) green fluorescent protein (GFP) (Mr of 30 kD) (FIG. 10) used as a model protein; (ii) FITC-labelled potato virus X (PVX) CP (Mr 18-27 kDa) (FIG. 12) (iii) Dehydrofolate reductase fused to N-terminal 23-amino acids M2e epitope of human influenza virus A membrane protein M2. The sequence of M2e has remained conserved in all influenza A isolates (Fiers W., De Filette M., Birkett A., Neyrinck S., Min Jou W. (2004) “Universal” human influenza A vaccine. Virus Res. 103 (1-2); 173-176; Lamb R. A., Zebedee S. L., Richardson C. D. (1985) Influenza virus M2 protein is an integral membrane protein expressed on the infected-cell surface. Cell 40(3), 627-33). Therefore, the N-terminal M2e could be attractive for nanovaccine development. In the present invention, the M2e epitope was fused to dihydrofolate reductase containing the 5-arginines tail; (iv) The antigenic determinant A of Rubella virus E1 glycoprotein was used for composite formation with SPs; (v) three neutralizing epitopes (65-212 aa) of hemagglutinine (HA) of human influenza A virus were bound to the surface of SP-platforms (FIG. 13); (vi) The epitope consisting of 12 amino acids from the CP of plum pox virus was fused to the PVX CP and the fusion protein expressed in E. coli. It should be mentioned that the fluorescent microscopy images of different compositions were similar in appearance. Therefore, only three representative types of compositions were presented in FIGS. 10, 12 and 13, and a schematic representation of detection by fluorescent microscopy of antigen linked to the surface of “SP-antigen” complex was given in FIG. 11.

The present invention showed that SPs were capable of binding entire molecules of GFP (Mr of 30 kD) and CP of PVX (Mr of 25 kDa) to their surface. The data of fluorescent microscopy indicated that the whole surface of all SPs used for GFP binding was covered with fluorescent molecules. Our data showed that SP can be used as a nanoplatform for foreign epitopes presentation on its surface (FIG. 10, 12). Therefore these compositions could be regarded as the candidates for nanovaccine particles assembly.

All types of SPs are water insoluble and produce either a colloidal solution or relatively stable suspension (depending on SPs size), Apparently, this state of denatured TMV CP is favourable for absorption of proteins on the SPs surface. Assembly of several composition listed in the invention was possible due to the unique capacity of SPs—to bind various functionally unrelated proteins.

The procedure of SP-based compositions assembly involves short incubation of SPs with the protein/epitope of interest and subsequent washing of complexes by low-speed centrifugation, the pellet resuspension and the complex stabilization by formaldehyde fixation (10 min at room temperature). The formaldehyde excess was removed upon washing SPs by centrifugation (10,000 g).

The present invention indicates that antigens/epitopes linked to the surface of SPs by this means retained their antigenic specificity. It was shown that foreign antigens linked to SPs reacted specifically with homologous (primary) antibodies during fluorimetric analyses of “SP-antigen/epitope” complexes (schematically illustrated by FIG. 11).

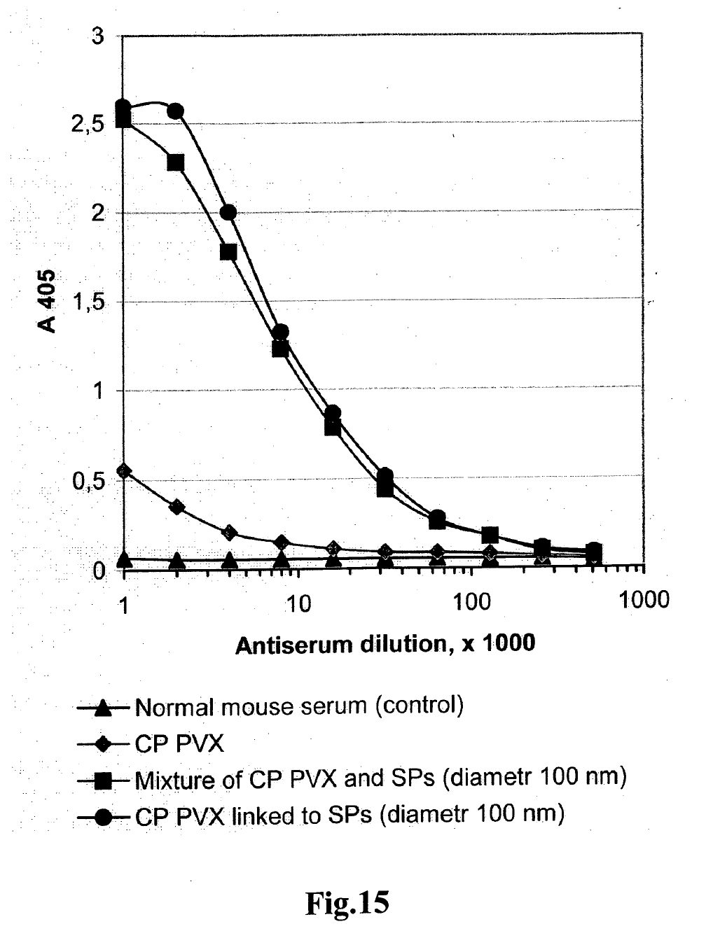

In a series of experiments, the “SP-PVX CP” complexes representing PVX CP linked to the surface of SPs by formaldehyde were used to immunize the mice. In control immunizations, the PVX CP alone was used. It was found that the titers of anti-PVX antibodies induced by “SP-PVX CP” complexes were considerably higher than those induced by the PVX CP. These data taken together provided direct evidence that specificity of foreign antigen linked to the surface of SP nanoplatform was retained and activity increased (FIG. 15).

Stimulation of the Immune Response (Adjuvant Effect) by SPs.

It has been reported that Papaya mosaic virus exhibits the booster activity and serves as adjuvant stimulating the immune response to foreign antigen (Leclerc et al., Adjuvant viral particle. U.S. Pat. No. 7,641,896, Jan. 5, 2010).

In the present invention, immune responses of native TMV and SPs were compared in trials to laboratory mice. The results of indirect ELISA showed that the SPs were significantly more immunogenic in mice than native TMV. Specific antibody response to SPs was up to 20-times higher than antibody response to TMV FIG. 8.

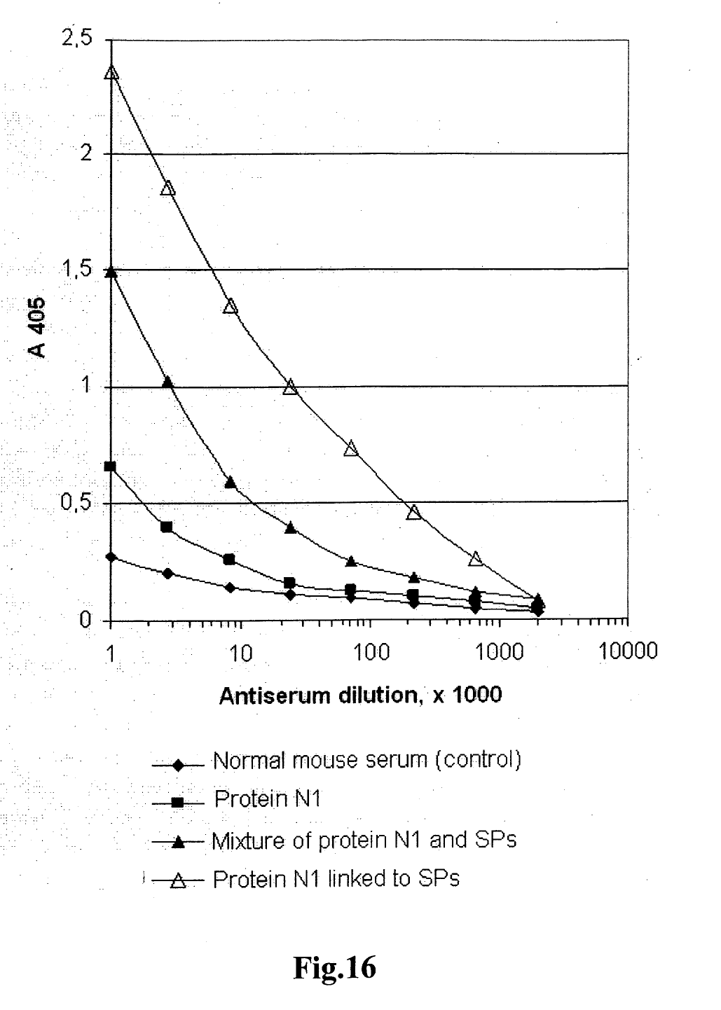

The present invention shows that SP-platforms have immunopotentiating or adjuvant properties, being used either in the form of mixture with antigen or in the form of composition comprised SPs-adjuvant and antigen covalently bound to its surface by formaldehyde. Two antigens were used as controls in the absence of adjuvant SPs, namely, PVX CP and the recombinant protein N1, which represented a fusion of N-deleted PVX CP with epitope (SMLNPIFTPA) from plum pox virus CP. The adjuvant activities of (i) mixtures “SPs+PVX CP” and “SPs+recombinant protein N1” and (ii) of compositions “SPs-PVX CP” and “SPs-N1” stabilized with formaldehyde were examined. The results presented in FIGS. 15 and 16 indicate that SPs particle platforms exhibited immunopotentiating or adjuvant properties.

### EXAMPLES

### Example 1

### Native Tobamoviruses and Different Forms of TMV CP Aggregates

TMV U1 strain was isolated from systemically infected Nicotiana tabacum L. cv. Samsun plants as described previously (Novikov, V. K. and Atabekov, J. G. (1970) A study of the mechanism controlling the host range of plant virus. I Virus—specific receptors of Chenopodium amaranticolor. Virology 41, 101-107).

Cucumber green mottle mosaic virus (CGMMV) was isolated from the extracts of systemically infected cucumber plants, clarified at 10,000 g, emulgated with chlorophorm (30 min) and centrifuged at 400 rpm for 30 min. Then polyethileneglicol (PEG 6000) was added (3%) to supernatant with 0.2M NaCl and incubated for night at 40%. After centrifugation at 10,000 g the pellet was resuspended in 0.02 M Tris-HCl, 0.001M EDTA, pH 7.5 and clarified at 10,000 g. The extraction from the pellet was repeated twice. The supernatants were mixed and subjected to two cycles of differential centrifugation (for 2 h at 45,000 rpm in Spinco L-50 rotorTi-50), Similar procedures were used for purification of other tobamoviruses, including Sunn-hemp mosaic virus (SHMV or Dolichos enation mosaic virus, DEMV), tomato mosaic virus (ToMV) and crucifer infecting tobamovirus (crTMV or TVCV). The 260/280 nm ratio of the virus preparations used was of 2.2. The preparations were examined by TEM in Hitachi-7.

The TMV CP was isolated by acetic acid (Fraenkel-Conrat, H. (1957) Degradation of tobacco mosaic virus with acetic acid. Virology 4, 1-4). The A protein was obtained in 10 mM Tris-HCl at pH range from 7.8 to 8.0. The TMV CP disk-like aggregates were obtained in 50 mM Na-phosphate buffer, pH 7.0; the CP reassembly into the helical form (VLP) was performed in 100 mM Na-phosphate buffer, pH 5.8-6.0 at room temperature overnight. Production of disk-like aggregates and VLP was controlled by TEM.

### Example 2

### Generation of SPs and their Examination by Transmission and Scanning Electron Microcopy

Heating of TMV and viral RNA-free CP preparations was performed in the “Tercyc” thermocycler (“DNA-technology”, Russia) for 10 sec at the temperature required. The process of the native TMV into SP transition was examined by TEM and SEM. The specimens were prepared as reported previously (Kaftanova A. S., Kiselev N. A., Novikov V. K., Atabekov J. G. (1975) Structure of products of protein reassembly and reconstitution of potato virus X. Virology 65, 283-287). The samples were viewed using a JEOL JEM-1011 microscope (JEOL, Japan) operating at 80 kV. Digital images were captured using a Gatan Erlangshen ES500W camera and Gatan Digital Micrograph™ software. For SEM, a drop of the sample was placed on a specimen stub and dried in air, then sputtered with gold with palladium in an ionic sputtering installation IB-3 Ion Coater (Eico, Japan), and examined in microscope JSM-6380LA (JEOL, Japan). FIG. 1-7 presents electron microphotographs illustrating a two-phase thermal remodeling of TMV.

### Example 3

### SPs Generation by RNA-Free Preparations of TMV CP

FIG. 8 presents a schematic (not to scale) representation of the SPs generation by native TMV and by RNA-free forms of TMV protein. The numbers indicate concentrations of native TMV (at left) and RNA-free TMV proteins (at right) heated at 94° C. or 65° C., respectively. The size ranges of SP (in nm) are indicated.

### Example 4

### Bacterially Expressed Proteins/Epitopes

The bacterially expressed recombinant antigens/epitopes used in the present invention for binding to the surface of SP platforms are described below. All recombinant DNA procedures were carried out by standard methods (Sambrook, J., Ftitsch, E. F. and Maniatis T. (1989) Molecular cloning: A laboratory Manual, 2nd edn. Cold Spring Harbor Laboratory, Cold Spring Harbor, N.Y.). E. coli strains DH5ot and M15[pREP4] were used for the cloning of created constructs and for superexpression, respectively.

Recombinant constructs expressing fusion (His)6 proteins were generated by cloning the PCR-amplified fragments into the pQE plasmid vector. Restriction fragments were ligated into the corresponding sites of the expression vector as described (Ivanov, K. I., Ivanov, P. A., Timofeeva, E. K., Dorokhov, Yu. L., and Atabekov, J. G. (1994) The immobilized movement proteins of two tobamoviruses form stable ribonucleoprotein complexes with full-length viral genomic RNA. FEBS Lett. 346, 217-22) Escherichia coli strain M 15 transformed with the recombinant vector was grown at 37° C. in liquid culture until an OD600 of 0.8-0.9 was reached. Expression of the proteins were induced with 1 mM IPTG followed by growth for 3 h at 37° C. The purification of proteins from cultures followed a general procedure described by the manufacturer (QIAGEN) for denaturing Ni-NTA chromatography.

### Example 5

### SPs-GFP Composites Formation; Fluorescent Microscopy

Generally, the SPs were incubated with foreign antigen/epitope in water at room temperature for 10 min “SPs-foreign antigen” complex formation occurred due to electrostatic or/and hydrophobic interactions. Furthermore, the generated complexes were centrifuged at 10,000 g to remove unbound antigen, the pellet consisting of “SPs-foreign antigen” complex was resuspended and then stabilized by 0.05% formaldehyde (10 min at room temperature). The formaldehyde excess was removed by SPs centrifugation (10,000 g).

Bacterially expressed green fluorescent protein (GFP) contained 6 His residues at the N-terminus, and 5 residues of Arg at the C-terminus was isolated as described previously (Karpova O. V., Ivanov K. I., Rodionova N. P., Dorokhov Yu. L., Atabekov J. G. (1997) Nontranslatability and dissimilar behavior in plants and protoplasts of viral RNA and movement protein complexes formed in vitro. Virology. 31, 230(1), 11-21; Zayakina O. V., Arkhipenko M. V., Kozlovsky S. V., Nikitin N. A., Smirnov A. V., Susi P., Rodionova N. P., Karpova O. V. and Atabekov J. G. (2008) Mutagenic analysis of Potato Virus X movement protein (TGBp1) and the coat protein (CP): in vitro TGBp1-CP binding and viral RNA translation activation. Molecular Plant Pathology 9 (1), 37-44). The ratio of GFP to SPs required for the complex formation was determined by gradual addition of GFP (50 μg/ml) to SPs (100 μg/ml) untill the aggregation of SPs was detected by dinamic light scattering.

The recombinant GFP was used as a model antigen contained 6 His residues at the N-terminus, and 5 residues of Arg at the C-terminus.

| | |
|---|---|
| 1 | ATGAGAGGATCTCACCATCACCATCACCATACGGATCCGCATGCGAGCTCGGTACCGAAT |
| 1 | M R G S H H H H H H T D P H A S S V P N |
| 61 | TCCATGGTGAGTAAAGGAGAAGAACTTTTCACTGGAGTTGICCCAATTCTTGTTGAATTA |
| 21 | S [symbol] V S K G E E L F T G V V P I L V E L |
| 121 | GATGGTGATGTTAATGGGCACAAATTTTCTGTCAGTGGAGAGGGTGAAGGTGATGCAACA |
| 41 | D G D V N G H K F S V S G E G E G D A T |
| 181 | TACGGAAAACTTACCCTTAAATTTATTTGCACTACTGGAAAACTACCTGTTCCATGGCCA |
| 61 | Y G K L T L K F I C T T G K L P V P W P |
| 241 | ACACTTGTCACTACTTTCTCTTATGGTGTTCAATGCTTTTCCCGTTATCCGGATCATATG |
| 81 | T L V T T F S Y G V Q C F S R Y P D H M |
| 301 | AAACGGCATGACTTTTTCAAGAGTGCCATGCCCGAAGGTTATGTACAGGAACGCACTATA |
| 101 | K R H D F F K S A M P E G Y V Q E R T I |
| 361 | TCTTTCAAAGATGACGGGAACTACAAGACGCGTGCTGAAGTCAAGTTTGAAGGTGATACC |
| 121 | S F K D D G N Y K T R A E V K F E G D T |
| 421 | CTTGTTAATCGTATCGAGTTAAAAGGTATTGATTTTAAAGAAGATGGAAACATTCTCGGA |
| 141 | L V N R I E L K G I D F K E D G N I L G |
| 481 | CACAAACTCGAGTACAACTATAACTCACACAATGTATACATCACGGCAGACAAACAAAAG |
| 161 | H K L E Y N Y N S H N V Y I T A D K Q K |
| 541 | AATGGAATCAAAGCTAACTTCAAAATTCGCCACAACATTGAAGATGGATCCGTTCAACTA |
| 181 | N G I K A N F K I R H N I E D G S V Q L |
| 601 | GCAGACCATTATCAACAAAATACTCCAATTGGCGATGGCCCTGTCCTTTTACCAGACAAC |
| 201 | A D H Y Q Q N T P I G D G P V L L P D N |
| 661 | CATTACCTGTCGACACAATCTGCCCTTTCGAAAGATCCCAACGAAAAGCGTGACCACATG |
| 221 | H Y L S T Q S A L S K D P N E K R D H M |
| 721 | GTCCTTCTTGAGTTIGTAACTGCTGCTGGGATTACACATGGCATGGATGAGCTCTACAAA |
| 241 | V L L E F V T A A G I T H G M D E L Y K |
| 781 | AGGAGAAGGAGACGTTAATCTAGA |
| 261 | R R R R R * |

Constructed on the base pQE31

bold type mark—6His

italic bold type mark—GFP start codon

underlined bold mark—5Arg

The five residues of Arg were added into recombinated molecule in order to increase GFP total positive charge. Electrostatic interaction between model antigen and SPs could be expected. The SPs (50 μg) were incubated with GFP (1 μg) in water and complex formation were registrated by fluorescent microscope. For detecting of fluorescence of GFP on the surface of the SPs 100 μl suspension of the SPs with GFP was incubated for 2 h at room temperature onto poly-1-lysine-coated-coverslips. After incubation the samples were fixed with 4% paraformaldehyde for 15 min at room temperature. The fixed samples were washed with water three times for 5 minutes. Then all samples were stained with 1,4-diazabicyclo-[2,2,2]octane (DABCO) (Sigma) anti-fading mounting media for 30 min at 4° C. The results were analysed using a Axiovert 200M fluorescence microscope (Carl Zeiss, Germany) equipped with a digital cooled camera ORCAII-ERG2 (Hamamatsu, Japan).

### Example 7

The antigenic determinant A of Rubella virus E1 glycoprotein was used for composite formation with SPs. Amino acid sequences of glycoprotein E1 with domain A (32 amino acid residues) is presented below:

EEAFTYLCTAPGCATQAPVPVRLAGVRFESKIVDGGCFAPWDLEATGAC
ICEIPTDVSCEGLGAWVPAAPCARIWNGTQRACTFWAVNAYSSGGYAQL
ASYFNPGGSYYKQYHPTACEVEPAFGHSDAACWGFPTDTVMSVFALASY
VQHPHKTVRVKFHTETRTVWQLSVAGVSCNVTTEHPFCNTPHGQLEVQV
PPDPGDLVEYI [symbol] [symbol] [symbol] [symbol] [symbol] [symbol]
PDCSRLVGATPERPRVDADDPLLRTAPGPGEVWVTPVIGSQARKCGLHI
RAGPYGHATVEMPEWIHAHTTSDPWHPPGPLGLKFKTVRPVALPRTLAP
PRNVRVTGCYQCGTPALVEGLAPGGGNCHLTVNGEDLGAVPPGKFVTAA
LLNTPPPYQVSCGGESDRATARVIDPAAQSFTGVVYGTHTTAVSETRQT
WAEWAAAHWWQLTLGAICALPLAGLLACCAKCLYYLRGAIAPR

Domain A is marked by italic bold type.

The recombinant protein (about 21-22 kD) contained the 6 His residues at the N-terminus, and four repeat of E1 glycoprotein domain A and was constructed on the base pQE-BT-4A. The polyepitope A (four repeats of E1 glycoprotein domain A) composed of SP complex was detected by the method of immunofluorescence schematically illustrated by FIG. 5.

For immunostaining the complex SPs-polyepitope A was incubated for 2 h at room temperature onto poly-1-lysine-coated coverslips. Before immunostaining the samples were fixed with 4% paraformaldehyde (PFA) for 15 min at room temperature. The fixed samples were washed with water three times (5 min each time). After washing, the samples were preincubated for 30 min with 1% bovine serum albumin (BSA) and 0.05% Tween-20 in phosphate-buffered saline (PBS, 7 mMNa₂HPO₄, 1.5 mM KH2PO4, pH7.4, 137 NaCl, 2.7 mM KCl) and then incubated for 30 min at room temperature in a humid chamber with mice antibodies against E1 glycoprotein of rubella virus in PBS supplemented with 1% BSA and 0.05% Tween-20. In control experiments, the samples were incubated without antibodies in a buffer containing 1% BSA and 0.05% Tween-20 in PBS. After incubation, the samples were washed three times (5 min each time) with washing buffer composed of PBS supplemented with 0.25% BSA and 0.05% Tween-20. Then, primary antibodies bound to antigens were revealed using Alexa 488-conjugated antimouse IgG (Molecular Probes, Eugene, Oreg.) during 30 min at room temperature. After binding the samples were washed three times (5 min each time) with washing buffer and one time with PBS. Then all samples were stained with 1,4 Diazabicyclo-[2,2,2]octane (DABCO) (Sigma) anti-fading mounting media 30 min at 4° C. The results of immunostaining were analysed using a LeicaDRMB fluorescence microscope equipped with a charge-coupled device camera.

### Example 8

### Linking of HDFR-M2e Fusion with the Surface of SPs

Next, was the recombinant protein consisting of dehydrofolate reductase (DHFR) fused with 23 amino acids long epitope M2e of Influenza-A virus (J. Fan, X. Liang, M. Horton, H. Perry, M. Citron, G. Heidecker, T. Fu, J. Joyce, C. Przysiecki, P. Keller (2004) Preclinical study of influenza virus A M2 peptide conjugate vaccines in mice, ferrets, and rhesus monkeys. Vaccine 22, 2993-3003) was used for “SP-epitope M2e” complex formation.

The ability of anti-M2 monoclonal antibody (Mab) to reduce viral replication implicates M2, in particular M2e, as a vaccine target. (Tompkins S. M., Zhao Z-Sh., Lo, Ch-Yu., Misplon J. A., Liu, T., Ye, Z., Hogan, R. J., Wu, Zh., Benton, K. A., Tumpey, T. M. and Epstein S. L. (2007) Matrix Protein 2 Vaccination and Protection against Influenza Viruses, Including Subtype H5N1. Emerg Infect Dis. 13, 426-135).

In the present invention, DHFR served as a carrier for the fusion with M2e epitope (DHFR-M2e):

MRGSHHHHHHGSGIMVRPLNSIVAVSQNMGIGKNGDLPWPPLRNEFKYF
QRMTTTSSVEGKQNLVIMGRKTWFSIPEKNRPLKDRINIVLSRELKEPP
RGAHFLAKSLDDALRLIEQPELASKVDMVWIVGGSSVYQEAMNQPGHLR
LFVTRIMQEFESDTFFPEIDLGKYKLLPEYPGVLSEVQEEKGIKYKFEV
YEKKGSRSSLLTEVETPIRNEWGCRCNDSSDKLN

Constructed on the base pQE 40

bold type mark—6His

underlined bold type mark—M2e peptide

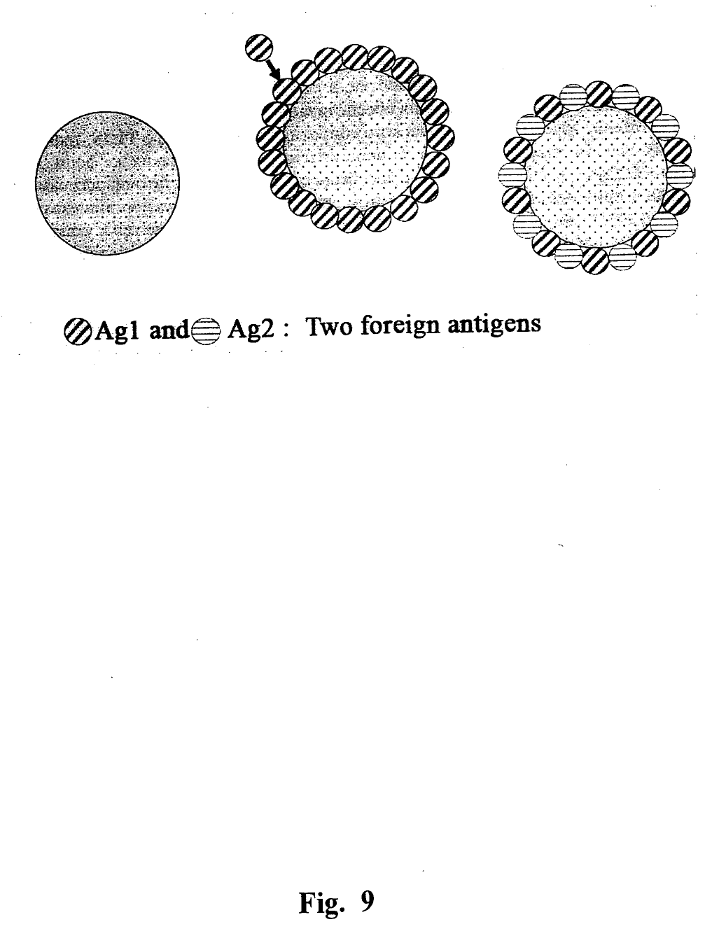

To determine the M2e peptide landing on the SNPs surface the method of immunofluorescence illustrated schematically by FIG. 9 was employed with mice antibodies against M2e peptide Influenza-A virus and chicken secondary antibodies conjugated with Alexa 488 (green). The M2e epitopes bound to SPs were antigenicflly active and the DHFR-M2e peptide ensured presentation of the foreign epitope on the surface of SPs particle platforms.

### Example 9

### Composition Comprising of SPs and FITC-Labelled PVX CP

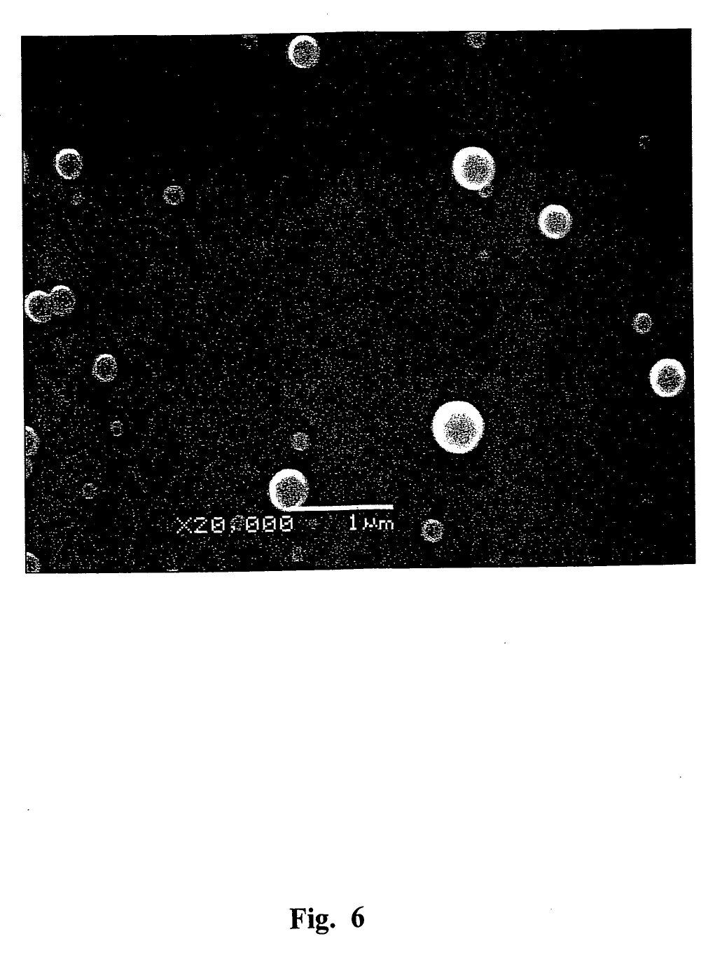

The PVX CP was isolated from purified PVX preparation in Tris-HCl, pH 7.5 by neutral LiCl, adjasting LiCl to 2M concentration. The mixture was exposed at −20° C. for night and centrifuged an 10,000 g. Supernatant was dializtd in threedistilled water and centrifuged at 100,000 g. Fluoresceine isothiocyanate (FITC) (18 μg) was added to 1 mg of PVX CP taken at concentration of 1 mg/ml, incubated for 24 h at 4° C. and dialized at water to remove the unbound label. The FITC-labelled PVX CP was used for “PSs-PVX CP” composition production. To this end 50 μg of PSs were incubated with 1 μg of CP in water. The complex was stabilized by 0.05% formaldehyde (10 min at, 20° C.). Production of SP-CP^{FITC} was detected FIG. 6 in fluorescent microscope (LeicaDRMB), supplied with integrated camera ORCAII-ERG2 (Hamamatsu, Japan).

### Example 10

### Recombinant Chimeric PVX CP Fused to Epitopes of Plum Pox Virus CP

Epitopes of CP PPV <<VNTNRDRDVDAG>> (12 residues) and <<SMLNPIFTPA>> (10 residues) (Fernandez-Fernandez, et al Fernández-Fernández M. R., Martinez-Torrecuadrada J. L., Roncal F., Dominguez E., Garcia J. A. (2002) Identification of immunogenic hot spots within plum pox potyvirus capsid protein for efficient antigen presentation. J. Virol. 76, 12646-12653) were fused with PVX CP into chimeric proteins. Epitope 12 was added to the N-end of full-length PPV Cp resulting in recombinant protein P2. Epitope 10 was added to the N-end of the CP PPV with first 20 amino acids deleted (protein N1). Recombinant constructions were obtained by cloning the PCR fragments in vector pQE as was described (Ivanov, K. I., et al. 1994. The immobilized movement proteins of two tobamoviruses form stable ribonucleoprotein complexes with full-length viral genomic RNA. FEBS Lett. 346, 217-220). All methods used farthermore were in accordance with (Sambrook, J., Ftitsch, E. F. and Maniatis T. (1989) Molecular cloning: A laboratory Manual, 2nd edn. Cold Spring Harbor Laboratory, Cold Spring Harbor, N.Y.). Purification of (H)₆ proteins on Ni-HTA-agarose was performed following (QIAGEN).

### Example 11

### Immunogenicity and Antigenic Specificity of SPs

To obtain mouse polyclonal antisera against SPs and TMV virions 6-8 week-old BALB/c mice were each immunized as described before (Erokhina et al., 2000). Antigenic properties of native TMV and SPs were compared by means of triple antibody sandwich ELISA (TAS-ELISA) using rabbit IgG specific to TMV or SPs as coating antibodies, polyclonal mouse antisera to the same antigens and normal mouse serum (negative control) as secondary antibodies and horseradish peroxidase-labelled anti-mouse IgG W402B (Promega) as detection antibodies. The DEAE-purified coating antibodies (1 μg/ml in carbonate).

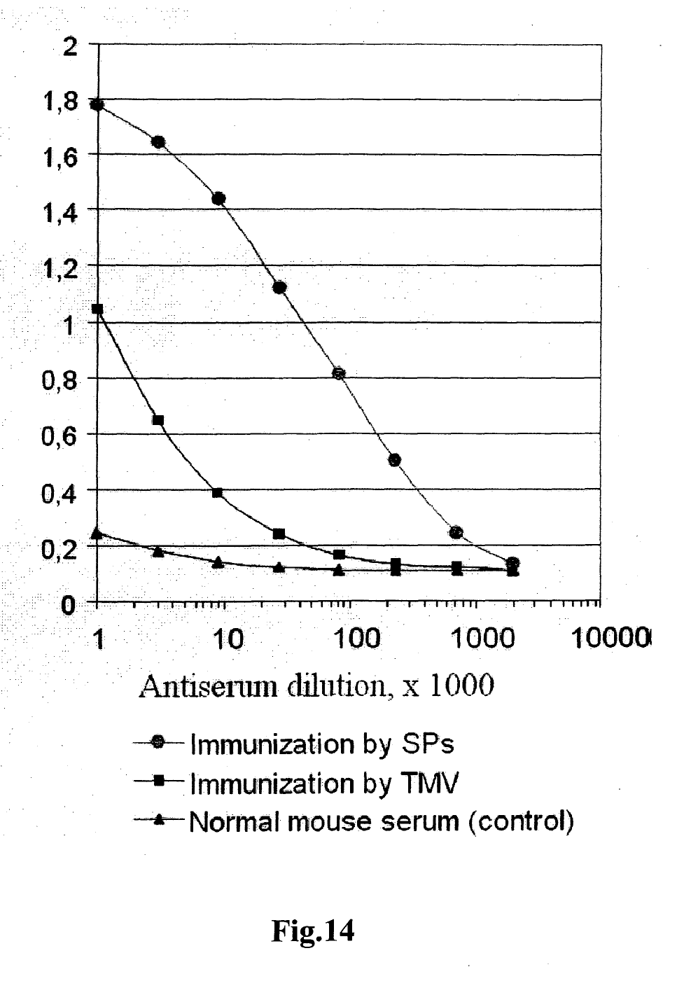

The results of indirect ELISA showed that the SPs were significantly more immunogenic in mice than native TMV. Specific antibody response to SPs was up to 20-times higher than antibody response to TMV FIG. 14.

Furthermore, the comparative analyses showed that native TMV and SPs were antigenically distantly related. Finally, antigenic specificity of SPs generated by different forms of TMV CP was examined. Namely, we compared the SPs generated by native TMV and by A protein. It was shown that SPs generated by these two distinct forms of TMV CP were antigenically closely related (Atabekov Joseph, Nikolai Nikitin, Marina Arkhipenko, Sergey Chirkov and Olga Karpova//Thermal transition of native TMV and RNA-free viral proteins into spherical nanoparticles. (2011) J Gen Virol; 92: 453-456).

1. Nanoparticles and microparticles (nano/microparticles) of diverse size but uniform shape, generated by heating, thermal denaturation and structural remodeling of a helical plant virus, wherein the morphology of said nano/microparticles is totally distinct from that of a virus, wherein said particles are water-insoluble, and wherein said nano/microparticles exhibit the capacity of platform-particles, that is, under certain conditions said particles bind to their surface foreign proteins/epitopes of interest.

2. Spherical nanoparticles (SNPs) and microparticles (SMPs) generated according to claim 1 by heating, thermal denaturation and structural remodeling of a helical plant virus, wherein said SNPs and SMPs exhibit the capacity of a particle-platforms, i.e. under certain conditions said particles bind to their surface foreign proteins/epitopes of interest.

3. The particles generated according to claim 1 by thermal denaturation of native TMV or other members of Tobamovirus genus, wherein the size of said SNP and SMP particles can be controlled by concentration of the virus used for heating, and wherein the said particles exhibit high level of stability and immunogenicity.

4. The SNPs and SMPs generated by heating, thermal denaturation and structural remodeling of different forms of RNA-free viral coat protein, including aggregates of protein subunits and virus-like particles, wherein said particles exhibit the capasity of a platform, i.e. under certain conditions bind to their surface foreign proteins/epitopes of interest.

5. Irregular shape nano/microparticles (IPs) of varying both of size and shape generated according to claim 1 by heating at a specific temperature dependent on the virus used, wherein subsequent heating of said IPs at 94-98° C. results in their transition into mature said SNP/SMP particle platforms.

6. The compositions comprising said SNP/SMP platform particles generated according to claim 1 by heating, thermal denaturation and structural remodeling of a helical plant virus, wherein said platform particles comprise foreign substance(s) linked to their surface, wherein these substances comprise proteins, including antigens, antigenic determinants (epitopes) pharmaceutical proteins, antibiotics, enzymes, synthetic polymers or inorganic substances, wherein said particle platforms can bind to their surface one or more types of different foreign substance(s) (e.g epitopes), and wherein said platform and foreign substance on its surface are linked by electrostatic, hydrophobic and/or covalent bonds.

7. Immunogenic composition obtained according to claim 6, wherein said composition comprises foreign proteins/immunogens/epitopes bound to the surface of said SNP/SMP platform particles, and wherein said foreign proteins/immunogens/epitopes are immunogenic within said composition.

8. A method of potentiating an immune response (adjuvant effect) against an antigen in an animal, wherein said adjuvant comprises said SNP/SMP platform particles generated according to claim 1 by heating, thermal denaturation and structural remodeling of a helical plant virus, and wherein said adjuvant comprises a mixture with said antigen, and wherein said adjuvant comprises the immunopotentiating composition of said SNP/SMP platform particles with said antigen linked to their surface.

9. The particles generated according to claim 2 by thermal denaturation of native TMV or other members of Tobamovirus genus, wherein the size of said SNP and SMP particles can be controlled by concentration of the virus used for heating, and wherein the said particles exhibit high level of stability and immunogenicity.

10. Irregular shape nano/microparticles (IPs) of varying both of size and shape generated according to claim 2 by heating at a specific temperature dependent on the virus used, wherein subsequent heating of said IPs at 94-98° C. results in their transition into mature said SNP/SMP particle platforms.

11. The compositions comprising said SNP/SMP platform particles generated according to claim 2 by heating, thermal denaturation and structural remodeling of a helical plant virus, wherein said platform particles comprise foreign substance(s) linked to their surface, wherein these substances comprise proteins, including antigens, antigenic determinants (epitopes) pharmaceutical proteins, antibiotics, enzymes, synthetic polymers or inorganic substances, wherein said particle platforms can bind to their surface one or more types of different foreign substance(s) (e.g. epitopes), and wherein said platform and foreign substance on its surface are linked by electrostatic, hydrophobic and/or covalent bonds.

12. Immunogenic composition obtained according to claim 11, wherein said composition comprises foreign proteins/immunogens/epitopes bound to the surface of said SNP/SMP platform particles, and wherein said foreign proteins/immunogens/epitopes are immunogenic within said composition.

13. A method of potentiating an immune response (adjuvant effect) against an antigen in an animal, wherein said adjuvant comprises said SNP/SMP platform particles generated according to claim 2 by heating, thermal denaturation and structural remodeling of a helical plant virus, and wherein said adjuvant comprises a mixture with said antigen, and wherein said adjuvant comprises the immunopotentiating composition of said SNP/SMP platform particles with said antigen linked to their surface.

14. Spherical nanoparticles (SNPs) and microparticles (SMPs) generated according to claim 2, wherein the helical plant virus is selected from the group consisting of, tobacco mosaic virus (TMV), other members of Tobamovirus genus, viruses belonging to family Flexiviridae, viruses belonging to Hordeivirus genus, and viruses from other structural and taxonomic groups,
